SEQUENCE LISTING (40 sequences)

SEQ ID NO: 1 - DNA, 3508 bp, Arabidopsis thaliana
cccgcacgaacgtttctggagtatctcttttctcttatctttcactccattacagagcaaagcttgactctttaagttcacatggtgtgttttaaagttccttcttttttactcaatttcgaagctttgtgtctgttttatcaaataatgatgagaaattgagaatgtgggtttttgtgactctttgattcgttgtttagagaattgggttttgggattttgtgttgaattgctcagttttgaagtcatgcagataatatgttcgatgatattcctgttagtgatgatggtgatgaaagtttcaggtttttctgactttgaagctcttttggagcttaagaaaggttttcagggtgacccttctcgaaaagttcttacttcatgggatgctaaagctttatcctctgataggtgtccgttaaactggtacggagttacttgtagtagcggaggtgttacttctattgatttgaatggtttcggtctgcttgggagtttcagttttccagtcattgttggtctgagaatgcttcagaatttgtcgattgcgaataatcagtttagtggaactctctctaacattggttcgttaacgtctcttaagtacttggatgtttcgggtaacttgttccatggcgcattaccttctggaattgagaatttgaggaaccttgagtttgtgaatctctctggtaacaacaacttgggaggagtgattccttctggttttgggagtcttgccaagttgaagtaccttgatttgcaaggcaacagtttctctggggaagtgatgagtctgttttcacagctaattagtgtggagtatgtagatataagcaggaacaacttctcgggttcgcttgatctgggtctagcaaagtcaagctttgtttcctcgattcgtcatttgaacgtaagtgggaattctttggtaggggagctatttgctcatgatggtatcccctttttcgatagcttagaggtgtttgatgctagcagcaatcaattgtctggttctgttccagtcttcagctttgttgtctctctcaagattcttcggttacaagacaatcagttgtcggcttctttaccaccgggccttctgcaagagagttcaaccatcttaactgaccttgatcttagcctcaatcaacttgaaggtcctatcggaagtataacctcttcaacactagagaagctcaatttgtcttcaaatagactatcgggatcattacctttgaaagttggacattgtgccattattgatctgagtaacaataagatatcaggagaattgtcaaggatacagaattggggagattctgtagaaattattcggttgagttcaaactcactcactggaacactacctggccaaacttctcaattcctgaggctgacttcactcaaggctgctaacaactcgttgcagggtgtactaccatttattttaggaacgtatcctgagctgaaagagattgatctcagtcacaaccagcttagtggcgtcataccgagtaatctgttcatatctgcaaagttgacggaactcaacttgtcaaacaataatttctctggttctcttcctcttcaagatgcaagtactgtgggaaaccttagcttgaccaatattggtctgtctcataactcgttaggtggagttctctcagaggaactaactcgcttccacaatctgatttctcttgacctttcttataacaactttgaagggaatatccctgatggccttccagacagcctaaagatgttcactgtctctgccaataatctctctggaaatgtgccggagaatcttagacggtttccagattcagcgtttcatccagggaacgctttactgaatgtccccatttctctaccaaaagataaaacagatataaccttacggaagcatgggtatcatatgaagacttctgtaaaggcagccctgatcataggcttggtcgtgggtactgctctacttgctcttgtttgcgtaatgtttcattttatgttgagaaagcaacatgatgaagagaagtcggatgtgactggagaaaagagtattgttccaaaaactgaaccttcttcttccaatgtgattgcagcaaagaacagtgtacaggaaaatgaatcttcgtcttctactacttctactccctcaatcaaggccaagctaccggtttcttcttcacgtttctcccaatatagtgactctgagaattcttcaccatttttgaaagaacctaacgaggagcttcactctgaatccagaaaagatgaaatcttgtcatcccaagtatcttcttcaactccatcgctgcctaaaatccagaattcaccagataatcctacgtctcgccagacttctatgagattggatgggaacctgtatattttcgacagttctcttaagcttactgcagaggaactgtctcgtgctccggctgaagctatcggaaggagttgccatggaacgctgtacagggcagtgcttaattctgattctgtattagcggtcaaatggttaagagaagggactgcaaaaggtaagaaggaatttgcaagggagataaagaaacttgggaacatcaatcacccaaatcttgtttctcttcaggcttactattggggtccaaaagaacacgagaagctcatcatatcaagatacatggatgcaccctgtttagctttctatctccaagaggctggacagctaaacctcccaccattgttactggaaaaccgtcttaaaattactctggacatagccagttgtcttagttacctgcacaatggggaagcaattccacacgggaatctaaaatcaactaatgttctcctgaaacctcctgaactcactgcacacctaacagattatagtcttcatcgattaatcacaccagaagccacgtcagagcaagtcttgaacgcagctgcccttggctactgccctccagagtttgccagctcgagtaaaccgtacccttctctgaaaagcgatgtctatgcctttggagtcattttgttagagcttctaacaggaaaagtttcgggagatatagtgtgcagtgatccaggtgtcgttgaactcactgaatgggttctactgcttgtgggacaaaaccgtgcaactgagtgttttgatccatcgattgttggatcacagggctcaaggaatccctttggggttcttactgatgtcttgcaggttgctctgagctgtatctcaccagcaccagagagaccggatatgaagctagtctctcaagagctctccagaattgttctcaagaggacagcaatctagaagagctttctttgtgaatgtcgctgtaaaaatgtcatgttggattcatttagttcttccattacccttttttatttgtagcataaaatgaaactgtaaatctaaatcaaatactccaattggttacgagacagtatagagggaacttcataaagagtaagaaaactcgtttggcttagcttagttgcttttcctttt

SEQ ID NO: 2 - protein, 1020 aa, Arabidopsis thaliana
MQIICSMIFLLVMMVMKVSGFSDFEALLELKKGFQGDPSRKVLTSWDAKALSSDRCPLNWYGVTCSSGGVTSIDLNGFGLLGSFSFPVIVGLRMLQNLSIANNQFSGTLSNIGSLTSLKYLDVSGNLFHGALPSGIENLRNLEFVNLSGNNNLGGVIPSGFGSLAKLKYLDLQGNSFSGEVMSLFSQLISVEYVDISRNNFSGSLDLGLAKSSFVSSIRHLNVSGNSLVGELFAHDGIPFFDSLEVFDASSNQLSGSVPVFSFVVSLKILRLQDNQLSASLPPGLLQESSTILTDLDLSLNQLEGPIGSITSSTLEKLNLSSNRLSGSLPLKVGHCAIIDLSNNKISGELSRIQNWGDSVEIIRLSSNSLTGTLPGQTSQFLRLTSLKAANNSLQGVLPFILGTYPELKEIDLSHNQLSGVIPSNLFISAKLTELNLSNNNFSGSLPLQDASTVGNLSLTNIGLSHNSLGGVLSEELTRFHNLISLDLSYNNFEGNIPDGLPDSLKMFTVSANNLSGNVPENLRRFPDSAFHPGNALLNVPISLPKDKTDITLRKHGYHMKTSVKAALIIGLVVGTALLALVCVMFHFMLRKQHDEEKSDVTGEKSIVPKTEPSSSNVIAAKNSVQENESSSSTTSTPSIKAKLPVSSSRFSQYSDSENSSPFLKEPNEELHSESRKDEILSSQVSSSTPSLPKIQNSPDNPTSRQTSMRLDGNLYIFDSSLKLTAEELSRAPAEAIGRSCHGTLYRAVLNSDSVLAVKWLREGTAKGKKEFAREIKKLGNINHPNLVSLQAYYWGPKEHEKLIISRYMDAPCLAFYLQEAGQLNLPPLLLENRLKITLDIASCLSYLHNGEAIPHGNLKSTNVLLKPPELTAHLTDYSLHRLITPEATSEQVLNAAALGYCPPEFASSSKPYPSLKSDVYAFGVILLELLTGKVSGDIVCSDPGVVELTEWVLLLVGQNRATECFDPSIVGSQGSRNPFGVLTDVLQVALSCISPAPERPDMKLVSQELSRIVLKRTAI

SEQ ID NO: 3 - DNA, 2282 bp, Arabidopsis thaliana
aactcaaagtccttacctttcccttctctaacctctctttctctttctctgtctctttgtgagaaatgggcatggaagctttgagatttcttcatgttatcttcttctttgtgctaattcttcactgtcattgtggaacatctctctctggttcttctgatgtgaagcttcttttaggaaaaatcaagtcttcactacaaggaaacagtgagagcttactgttgtcttcttggaactcctctgttcctgtttgtcaatggagaggtgtaaaatgggtattttcaaatgggtctcctcttcaatgtagtgacctctcttcaccacaatggactaatacctctctgttcaacgactcttctcttcaccttctctctcttcagcttccttctgctaatctcactggttcactccctagagagattggtgagttctctatgcttcaaagtgtgttcctcaacatcaattcattaagtgggtcaatccctcttgagcttggttacacttcttctctctctgatgttgatttgagtggtaatgccttagctggggttttgcctccatcgatttggaacctctgtgataagcttgtctctttcaagattcatggtaataacttgtctggggttttgcctgagcctgctttgccaaattcgacttgtggtaatctccaagttcttgatttgggtggtaataagttctcaggtgagtttcctgagtttataactaggtttaaaggtgtgaagtcacttgatctttcaagtaatgtctttgaaggtcttgttcctgagggtttaggtgtattagagctagaaagtctcaatctttctcataataacttcagtgggatgttgccagattttggtgagtcaaagtttggagcagaatctttcgaagggaacagtcctagcctttgtggtttgcctttgaagccttgtctaggctcctctaggttaagtccaggtgctgttgctggtctggtgattggtttaatgtctggagctgttgttgtggcctcgttgttaatagggtatttgcagaacaagaaaagaaagagtagtatagagagtgaagatgatttggaagaaggtgatgaagaagatgaaatcggtgagaaagaaggcggtgaaggaaagttagttgtgtttcaaggtggtgagaatctgacgttggatgatgttttgaatgctactgggcaagttatggagaagactagctatggtactgtctataaagctaagcttagtgatggagggaatattgcattgaggctattgagagaaggtacttgcaaggatagaagttcttgtctgcctgttataaggcagttaggacgcattcggcatgagaatttggttcccttgagagctttctatcaagggaagagaggagaaaagcttctcatctatgactatcttcccaacataagcttacatgatttgttgcatgaaagtaaacctcgaaagccagctttgaattgggctaggagacacaagattgcacttggaatagcgaggggacttgcttatcttcatactggacaagaagttcctatcatccatggaaatattagatcaaagaacgtgcttgtggacgactttttctttgcaaggctaactgagtttgggcttgacaagataatggtacaggcagtagcagatgagattgtctcgcaggcgaaatccgacgggtacaaagcacctgaactccacaagatgaagaaatgcaatccaaggagtgatgtttacgcctttgggatccttctcttggagatattgatgggtaagaaaccaggaaagagtggaaggaacggtaatgagtttgtggacttgccttctttggttaaagctgcggtgttggaagagacgacaatggaggttttcgacttggaggcaatgaaagggattaggagcccaatggaagaaggtttggttcatgcattgaagctagcgatgggatgttgtgctcctgttacaacagttagacccagcatggaagaggttgtgaagcagttggaagagaacagaccgaggaatagatccgcattgtacagcccaaccgaaaccaggagcgacgccgaaactccattttgagtctaattgtgttcttctacaagctttttttttcttctgtaattcctcactttagttggctatatttgtttccttggcaagaaaccattctgatttgattttatctatatttgttgatgagaaatggtgtaagtgtaacagaagcagttcaaagtattaatattgatagaagaatatatgatacatgatgat

SEQ ID NO: 4 - protein, 674 aa, Arabidopsis thaliana
MGMEALRFLHVIFFFVLILHCHCGTSLSGSSDVKLLLGKIKSSLQGNSESLLLSSWNSSVPVCQWRGVKWVFSNGSPLQCSDLSSPQWTNTSLFNDSSLHLLSLQLPSANLTGSLPREIGEFSMLQSVFLNINSLSGSIPLELGYTSSLSDVDLSGNALAGVLPPSIWNLCDKLVSFKIHGNNLSGVLPEPALPNSTCGNLQVLDLGGNKFSGEFPEFITRFKGVKSLDLSSNVFEGLVPEGLGVLELESLNLSHNNFSGMLPDFGESKFGAESFEGNSPSLCGLPLKPCLGSSRLSPGAVAGLVIGLMSGAVVVASLLIGYLQNKKRKSSIESEDDLEEGDEEDEIGEKEGGEGKLVVFQGGENLTLDDVLNATGQVMEKTSYGTVYKAKLSDGGNIALRLLREGTCKDRSSCLPVIRQLGRIRHENLVPLRAFYQGKRGEKLLIYDYLPNISLHDLLHESKPRKPALNWARRHKIALGIARGLAYLHTGQEVPIIHGNIRSKNVLVDDFFFARLTEFGLDKIMVQAVADEIVSQAKSDGYKAPELHKMKKCNPRSDVYAFGILLLEILMGKKPGKSGRNGNEFVDLPSLVKAAVLEETTMEVFDLEAMKGIRSPMEEGLVHALKLAMGCCAPVTTVRPSMEEVVKQLEENRPRNRSALYSPTETRSDAETPF

SEQ ID NO: 5 - DNA, 2919 bp, Arabidopsis thaliana
ataattagtcttttgctacaacactcactcactcacttatttccattacctctctcactaaaaatcaaacttttctctgtctcactctctccaaaaacagaggaaggagaacaaaccaagaactcaagctcccctgctcttctcatctttcttcttcgttttctattttctttcttctttattctttgattcttttgaaatgaatcatctttacaaaaacccatttcgcatttatgaaattagtttccatttctgcgcgagtctactcttgtgtttcttactcttctctgctcaagctgttgcaggtggtggaggtggtggtcactcttgggatggaatcgtggtgactcaggcgaactatcaggcactccaagctatcaaacacgaactcattgacttcaccggagttctcaaaagctggaacaactctgcctcctctcaagtttgctccggctgggctggaatcaaatgcctccgaggtcaagtggtcgccattcagctcccttggaagggacttggcggtactatctctgagaagattggacagcttggaagtctcagaaaacttagcctccacaacaatgttatcgccggttcggttcctcgttcccttggctacctcaagagcctccgtggagtctatctcttcaacaatcgtctctcgggttccatcccagtctcactgggtaactgccctcttctccagaatcttgatctcagcagtaaccagctaactggagccatcccgcctagccttactgaatccacgaggctctataggctcaatctcagcttcaattcactttccggtccccttccggttagtgtagccagatcatataccctcacttttcttgatcttcagcataacaacctatctggttcgatacctgactttttcgtcaatggctcccaccctctcaaaacgctgaatcttgaccacaatcgcttctctggagctgttccagtgtctctctgcaagcatagtctgttggaagaggtttctataagccataaccagctctcaggttctattcccagagaatgtggaggtcttccacacctccagagcctggatttctcttacaattcaatcaacggaaccatcccagacagtttctccaacctttcatctctagtttcactaaatctcgaaagcaaccacctcaaaggcccaatcccagatgccattgatagacttcacaacctgacagagcttaacctgaagagaaacaagatcaatgggccaatcccagaaacaatagggaacatatctggaatcaaaaagcttgatctgtcagaaaacaacttcacaggtccaatcccgctctcactagtccacctggcgaagctttcttcattcaacgtctcctacaacaccctctctggtcctgttccacctgtcctctccaagaagttcaactcaagctctttcttgggcaacatccagctttgtggctacagctcctcaaatccatgccctgctcctgatcaccaccatcccctcaccctttcacctacctcatcacaagaaccaagaaaacaccaccacagaaaactctcagtgaaagacgtaatcctaatcgccatcggagctcttctagccatcctccttctactatgctgcatactcctctgctgcctgatcaagaaacgcgccgccttgaaacaaaaagatggaaaggacaagacctcagagaagacagtatcagcaggtgtcgcaggaacagcatcagcaggaggtgaaatgggaggcaagctagtccatttcgatggtccgttcgtgttcaccgccgatgatctgctctgtgccacagctgagatcatggggaaaagtacatatggcacagcatacaaggccacattagaagacggcaatgaagtcgctgtgaagcgtctgagagagaaaacaacaaaaggagtcaaagaattcgaaggagaagtcacagctttaggcaagattcgtcatcagaatcttcttgcactaagagcttactacttaggacctaaaggagagaagcttctcgtctttgattacatgtctaaaggaagcctctctgcatttcttcacgctcgaggaccagaaaccctaattccgtgggaaacaagaatgaagatagcaaaaggaatcagtcgtggtttagctcaccttcacagcaacgagaacatgatccatgagaatctcacagcaagcaacattctcctcgacgaacaaaccaacgcacacatcgccgattacggtctctcaagactcatgaccgccgccgcagcaacaaacgtaatcgcaaccgcaggaacattaggatacagagcaccggagttttcgaaaatcaaaaatgcaagcgctaagaccgatgtctacagcttaggaatcatcatattggagcttttgactgggaaatctccaggagagccgacgaatgggatggatttgccacaatgggtagcttcgattgtgaaagaagagtggactaatgaagtttttgatttggagctgatgagagagacacaaagcgttggtgatgagcttttgaatactttgaaattggctttacattgtgttgatccgtcgccggcggcgaggccagaagctaatcaggtggtggagcagttggaggagattaggccggagactgaagtggagacggagacgacgccgtttggatctggtggtgaaggaggtaaagatttagggtcaaatgaggagtaaaggaaagttttggggtttatgtgaaagagaataaagaagaagggaccgatatacaaataccacatttatattttcattgtttattctttgattcaggtaatttgtagataaaaatggtttgaagtttgggctagtaatgtaatgtaatttgattattcattcttttttggttgaagaagtaatacgagaattgatttggaccttactta

SEQ ID NO: 6 - protein, 836 aa, Arabidopsis thaliana
MNHLYKNPFRIYEISFHFCASLLLCFLLFSAQAVAGGGGGGHSWDGIVVTQANYQALQAIKHELIDFTGVLKSWNNSASSQVCSGWAGIKCLRGQVVAIQLPWKGLGGTISEKIGQLGSLRKLSLHNNVIAGSVPRSLGYLKSLRGVYLFNNRLSGSIPVSLGNCPLLQNLDLSSNQLTGAIPPSLTESTRLYRLNLSFNSLSGPLPVSVARSYTLTFLDLQHNNLSGSIPDFFVNGSHPLKTLNLDHNRFSGAVPVSLCKHSLLEEVSISHNQLSGSIPRECGGLPHLQSLDFSYNSINGTIPDSFSNLSSLVSLNLESNHLKGPIPDAIDRLHNLTELNLKRNKINGPIPETIGNISGIKKLDLSENNFTGPIPLSLVHLAKLSSFNVSYNTLSGPVPPVLSKKFNSSSFLGNIQLCGYSSSNPCPAPDHHHPLTLSPTSSQEPRKHHHRKLSVKDVILIAIGALLAILLLLCCILLCCLIKKRAALKQKDGKDKTSEKTVSAGVAGTASAGGEMGGKLVHFDGPFVFTADDLLCATAEIMGKSTYGTAYKATLEDGNEVAVKRLREKTTKGVKEFEGEVTALGKIRHQNLLALRAYYLGPKGEKLLVFDYMSKGSLSAFLHARGPETLIPWETRMKIAKGISRGLAHLHSNENMIHENLTASNILLDEQTNAHIADYGLSRLMTAAAATNVIATAGTLGYRAPEFSKIKNASAKTDVYSLGIIILELLTGKSPGEPTNGMDLPQWVASIVKEEWTNEVFDLELMRETQSVGDELLNTLKLALHCVDPSPAARPEANQVVEQLEEIRPETEVETETTPFGSGGEGGKDLGSNEE

SEQ ID NO: 7 - DNA, 2303 bp, Arabidopsis thaliana
atggagtttatcacccaaaaccaagctattacttctctgagtatgatcaacaccgacattgatcaacctaaggcttcacttcgaagccgatttctccttcatctaatcatatgcctcctcttcttcgtccctccctgctcaagccaagcctgggatggtgtagtgataacacaagctgattaccaaggtcttcaagcagttaaacaagaactgattgatccaagaggcttcttgagaagctggaacggttctggattcagtgcttgctctggaggctgggctggaataaaatgtgctcaaggtcaagtcatcgtcattcaacttccatggaagagtcttggaggtagaatctctgagaaaatcggacagcttcaagctcttcgtaagcttagtcttcatgacaacaaccttggaggttcgattccaatgtcattgggactcattcctaatcttagaggagttcagcttttcaacaaccgtctcactggctcaatccctgcttctctcggtgtatcacatttccttcaaacgcttgatctcagcaataacttgctctctgagattattccaccgaatcttgctgactcttctaagcttcttaggcttaatctcagctttaattcactgtctggtcaaatcccagtgagtctctctcggtcttcttcccttcagtttcttgctcttgaccataacaacctctctggtccaatcttagatacttggggtagtaagatccgtggaaccctaccctctgagctaagcaaactcactaagcttagaaaaatggatattagtggtaacagtgttagtggccatatccctgaaaccctagggaacatttcttctcttatacatttggatttgtctcaaaacaaactcaccggagagattcctatttcgatttctgacctggaaagcctgaacttcttcaatgtctcttataacaacttatctggtcctgttcctactctcttgtcccaaaagttcaattcctcttcttttgtcgggaactcactgctttgcggatacagcgtttcgacaccatgtcctactcttccttcaccgtctcctgaaaaagaaaggaaaccatcccacaggaatctgagtaccaaagacatcatcctcattgcgtctggagcactcctcatagttatgcttatccttgtttgtgttctatgttgcttgctgaggaagaaagccaatgaaaccaaagccaagggcggagaggctggacctggagctgtagccgcaaagactgagaaaggaggggaagctgaagctggaggtgaaactggagggaagctggttcattttgatggaccaatggcgtttactgcagatgatcttttgtgtgcaacagcagagataatggggaaaagcacttatgggactgtgtacaaagctacacttgaagatggaagtcaagtagcagtgaagagattgagagaaagatcaccaaaagtcaaaaagagagagaagcttgtcgtcttcgattacatgtctagaggaagccttgccacgtttctccatgcaagaggaccagatgtacatatcaattggcctacaaggatgagcttaataaagggaatggctcgaggcttgttctaccttcacacacacgcaaatatcatccacggaaacttaacatcaagcaatgtgcttttggatgagaacataaccgcgaagatctcagattacggtctctcaaggctaatgacagcagcagcaggatccagcgtgattgcaacagctggtgcgttaggttacagagcacctgagctctctaagctgaagaaagccaacacgaaaaccgatgtgtacagcctcggtgtgatcatattggaactgttgactgggaaatctccgagtgaggccttaaacggtgtggatttgcctcaatgggttgctactgcggttaaagaagagtggactaatgaggtttttgatttggagctgttgaatgatgtgaacacaatgggtgatgagattttgaatacactgaaactggctttacattgtgttgatgctacaccatcaacaagacctgaagctcaacaagtgatgacacaacttggagagattagaccggaagagacaacagcaacaacatcagaaccgttgattgatgtccctgaagcttctgcttccacaagtcaatagaaagagtggagaatttttgtagtgtggaaatgttgcagagtagtagttgatttcttttgcaattctttaattcttccaagttgtttttttgttctttgtttgtttcagtgttttgaaccatttcttcatcagtttatgtttgt

SEQ ID NO: 8 - protein, 719 aa, Arabidopsis thaliana
MEFITQNQAITSLSMINTDIDQPKASLRSRFLLHLIICLLFFVPPCSSQAWDGVVITQADYQGLQAVKQELIDPRGFLRSWNGSGFSACSGGWAGIKCAQGQVIVIQLPWKSLGGRISEKIGQLQALRKLSLHDNNLGGSIPMSLGLIPNLRGVQLFNNRLTGSIPASLGVSHFLQTLDLSNNLLSEIIPPNLADSSKLLRLNLSFNSLSGQIPVSLSRSSSLQFLALDHNNLSGPILDTWGSKIRGTLPSELSKLTKLRKMDISGNSVSGHIPETLGNISSLIHLDLSQNKLTGEIPISISDLESLNFFNVSYNNLSGPVPTLLSQKFNSSSFVGNSLLCGYSVSTPCPTLPSPSPEKERKPSHRNLSTKDIILIASGALLIVMLILVCVLCCLLRKKANETKAKGGEAGPGAVAAKTEKGGEAEAGGETGGKLVHFDGPMAFTADDLLCATAEIMGKSTYGTVYKATLEDGSQVAVKRLRERSPKVKKREKLVVFDYMSRGSLATFLHARGPDVHINWPTRMSLIKGMARGLFYLHTHANIIHGNLTSSNVLLDENITAKISDYGLSRLMTAAAGSSVIATAGALGYRAPELSKLKKANTKTDVYSLGVIILELLTGKSPSEALNGVDLPQWVATAVKEEWTNEVFDLELLNDVNTMGDEILNTLKLALHCVDATPSTRPEAQQVMTQLGEIRPEETTATTSEPLIDVPEASASTSQ

SEQ ID NO: 9 - DNA, 3176 bp, Arabidopsis thaliana
atggggcaacttccatcacaggacatcatggcattgcttgaattcaagaaaggcatcaaacatgaccctacagggtttgtccttaattcatggaacgatgagtctattgacttcaatggttgtccttcttcatggaatggtattgtctgcaatggcggtaatgtggctggtgttgttttggataatttgggtttgactgctgatgctgatttcagtctgttctccaacttgacaaagcttgtcaagctatctatgtcaaacaattccctttctggtgtgttgccaaatgatttaggcagtttcaaaagcctccagttcctggatttatcagataatctgttctcttcttcattgcctaaagagattggtagatcagtaagcttgaggaatctttctttatctggtaacaacttttctggtgagattccagagtctatgggtggtttgatttcacttcagtcgttggatatgagtagcaactccttatctggacctttgccaaagtccttgacaaggctgaacgatctgctgtacttgaatctgtcttctaatggattcacggggaaaatgccaaggggctttgagttgatttcaagtcttgaagtgcttgatttgcatggtaattcaattgatggtaatcttgatggggagtttttccttttaacgaatgcgagctatgtcgatatcagtggaaacagattggtgacgacgtctgggaaactgttacctggtgtttctgagagtatcaagcacttgaatctcagccataatcagcttgaaggttcattgactagtgggtttcagttgtttcagaacttaaaagtcttggatcttagctacaacatgttatctggagaattaccaggtttcaattatgtctatgatcttgaagttctcaagcttagcaacaacagattctcaggttcccttcccaataatttgttaaaaggtgactctttgcttttaacaacgctggatttaagtggcaacaatctctcagggccagtaagttctatcatgtcaactactcttcacacccttgatctttcttcaaattcactgaccggggagcttcctctcttgactgggggatgtgttttactcgatctctcaaacaaccagtttgaaggaaatctgacaagatggtcaaaatgggagaatatagagtaccttgatctaagccagaaccatttcactgggtcgttcccagacgcaactccccagcttctgcgagcaaatcatcttaatctttcctacaataagctcacaggctcacttccagaacggattccaactcactatccgaagcttcgggtacttgatataagttctaacagcttagaagggccaattccaggtgcgttactatccatgcccactttggaggaaatccaccttcaaaacaatggcatgacaggtaacatcggacccctgccttcttctggttccagaatccgtcttcttgatctttctcacaaccggtttgacggggatcttcctggtgtattcgggtctttaaccaatcttcaagtgctgaatctcgcagcaaataatttgtctggctctttgcctagctccatgaatgacattgtctctctaagctcattagacgtatcccagaatcatttcactggaccactcccaagcaacttatctagcaacattatggccttcaacgtgtcatataatgatctatcagggactgtgccagagaatctgaagaacttccctccgccttccttttatcctggaaacagcaagctcgtcttgcctgctgggtcccctggatcaagtgcatcagaagcttccaagaataaatccacgaacaaacttgttaaggttgtcataatagtttcttgcgctgttgctctaattattctcatccttgtggctatcctccttttctgcatctgcaaatcaagaaggcgcgaagaacgtagtattactggtaaagaaactaataggcgggctcaaactatcccctcaggcagcggaggtggtatggttgtctctgctgaggatctggttgcttcaagaaaaggctcatcatccgaaatcctcagtccagatgaaaaactagctgttgcaaccggcttctctccttctaagaccagcaatttgtcttggtcacctggctcaggagattcattcccagctgatcagcaactagcacggcttgatgtgaggtcaccagatagacttgtgggagagctgcattttctggatgattcaattaagttgactccagaggaattatcaagggcaccagctgaagttctgggaagaagtagccacggcacttcttacagggcaacgctggataatggagtgtttctaacagtgaaatggctaagagaaggagtggcaaagcagagaaaggagtttgctaaagaggtgaagaaattttctaacattagacatcctaatgttgtgactctccgaggagctgttccacacggtaacctcaaggcaacaaacattctcttagatggagcagagctaaacgcgcgtgtggcagattactgcctccaccgtctcatgacacaagcaggcacagtcgaacagattctagacgcaggaatcctcggttaccgagctcccgagctagcagcttcaagaaaaccattaccttctttcaaatcagatgtatatgcgtttggtgtgatacttcttgagatcctaaccggaagatgtgcaggagatgtaatcacaggtgagcaagaaggtgttgatctaaccgattgggttaggttacgtgttgcagaagggcgtggtgcagaatgctttgattcggtattgactcaggagatgggaagtgatccggtaacagaaaaaggcatgaaagaagttcttgggattgctttgaggtgtataagatcagtttctgagagacctggtattaagaccatttacgaagatctttcttctatttaggaacaatgtttttttttttttttatgttttgtaggttccaatagagtttggttcctctgaatgtgttctcctttgtcaagttcatgttctgtttcgggagaaacatgttcttgattcaaggtactagtgtgttgtgtcttctgttagtttactttacattttccctgtaaaaaaacgaaatctgctcatgtctcatgagtgaatgaaatttcattttttcccatttatattctctcttcttg

SEQ ID NO: 10 - protein, 977 aa, Arabidopsis thaliana
MGQLPSQDIMALLEFKKGIKHDPTGFVLNSWNDESIDFNGCPSSWNGIVCNGGNVAGVVLDNLGLTADADFSLFSNLTKLVKLSMSNNSLSGVLPNDLGSFKSLQFLDLSDNLFSSSLPKEIGRSVSLRNLSLSGNNFSGEIPESMGGLISLQSLDMSSNSLSGPLPKSLTRLNDLLYLNLSSNGFTGKMPRGFELISSLEVLDLHGNSIDGNLDGEFFLLTNASYVDISGNRLVTTSGKLLPGVSESIKHLNLSHNQLEGSLTSGFQLFQNLKVLDLSYNMLSGELPGFNYVYDLEVLKLSNNRFSGSLPNNLLKGDSLLLTTLDLSGNNLSGPVSSIMSTTLHTLDLSSNSLTGELPLLTGGCVLLDLSNNQFEGNLTRWSKWENIEYLDLSQNHFTGSFPDATPQLLRANHLNLSYNKLTGSLPERIPTHYPKLRVLDISSNSLEGPIPGALLSMPTLEEIHLQNNGMTGNIGPLPSSGSRIRLLDLSHNRFDGDLPGVFGSLTNLQVLNLAANNLSGSLPSSMNDIVSLSSLDVSQNHFTGPLPSNLSSNIMAFNVSYNDLSGTVPENLKNFPPPSFYPGNSKLVLPAGSPGSSASEASKNKSTNKLVKVVIIVSCAVALIILILVAILLFCICKSRRREERSITGKETNRRAQTIPSGSGGGMVVSAEDLVASRKGSSSEILSPDEKLAVATGFSPSKTSNLSWSPGSGDSFPADQQLARLDVRSPDRLVGELHFLDDSIKLTPEELSRAPAEVLGRSSHGTSYRATLDNGVFLTVKWLREGVAKQRKEFAKEVKKFSNIRHPNVVTLRGAVPHGNLKATNILLDGAELNARVADYCLHRLMTQAGTVEQILDAGILGYRAPELAASRKPLPSFKSDVYAFGVILLEILTGRCAGDVITGEQEGVDLTDWVRLRVAEGRGAECFDSVLTQEMGSDPVTEKGMKEVLGIALRCIRSVSERPGIKTIYEDLSSI

SEQ ID NO: 11 - DNA, 3693 bp, Arabidopsis thaliana
aaagaagtataagaaaactaatataaaaattcgatttttttttggaaaacctcaaaaacacactcttctctctctcttcttcttcttcccctctttcaatttttttcagattccgagtacaatccctaaatctccgtctctctgtttctctcttctctcaagtctcagctatgaatccttactagggtttattctttctgtgtttctcactctgcatttgcttctgtggagtagatagattcttcagggctaaatgagtcattttctcactttctgcttcctctcactccttctcctcctccacggtgccaacgccgtcaccgagacggagctccgatctctactcgagttccgtaaagggatccgtgacgagacctcacaccaacgaatctcatggtcagatacaagcagcctcactgatccatctacttgccctaatgactggcctggtatctcttgcgaccctgaaaccggttcaatcatcgccattaatctcgaccgtcgtggtctctccggcgaactcaaattcagcactctctctggtcttactcgtcttcgtaacctctctttatccggaaacagcttctcgggtcgggtcgttccttcattgggtggaatctcgtcgcttcaacacttagatctgtcggataacggtttctacggacctataccgggtcggatctcggagctttggagtttgaatcatctcaatttgtcttccaacaagttcgaaggtgggtttcctagtggctttcggaatcttcagcagctaagatctttggatttgcataagaacgagatttggggagatgttggtgagattttcactgagcttaagaatgttgagttcgttgatttgagctgtaaccgtttcaacggtggcttatctttaccaatggagaacatttccagcatttccaacactctccgtcacttgaatttgagccacaatgcgttaaatggcaagtttttcagtgaggagtccattggttcgttcaagaatctcgagattgttgacttagagaataaccagatcaatggtgagttaccgcattttggatcacagccgagtttgagaatattgaagcttgctcgtaacgagttgttcggtttagtacctcaagagttgttgcagagttcgattcctttgctagagctagatctaagtcgaaacggttttacaggatcaattagtgaaatcaattcatcaacgttaactatgttgaatctttcttctaacggcctgtctggggatcttccgtcgagttttaaaagttgctcagtgatagatttgagtgggaatactttctctggcgacgtgtctgttgttcaaaagtgggaagctactccagatgttcttgatcttagctcaaacaacttgtcgggaagcttgccgaactttacttctgcattttcaagattaagtgtgttgagcattaggaataattctgtctctggtagcttgccttctctgtggggtgattctcaattctctgtgattgatctcagttcaaataagttcagtggcttcattccagtgagtttcttcacttttgcaagcttgagaagtttgaatctttctaggaataacttggaggggccaattccttttcgaggttcacgtgcaagcgaacttttggttctaaattcttatccgcagatggagttacttgacctttcaaccaattctttgacgggtatgctgcctggggatataggtacaatggaaaaaattaaagtgctgaatcttgccaataataagctgtccggggaacttccaagcgacctcaacaagcttagtggccttttgttccttgacttgtcaaacaatactttcaagggtcaaattcccaataagcttccttcccaaatggtgggattcaatgtgtcgtataacgacctatcaggcataattccggaagaccttagaagctacccgccttcttcattttatcctggtaactccaagctaagtctcccaggacggattccagcggattcttctggtgatctgtctttacctgggaaaaaacatcattcgaagcttagcataagaatagcgattattgttgcttcagttggagctgctattatgattctttttgtcttgtttgcctatcatcgaacacagctcaaggactttcacgggaggaatagatttacagaccaagcaacgactagagacaccaagtttggacgctcttctcgtccctccttattcaacttcagctcaaatgttgagcagcagtcatcatctttgagcttctcaaacgatcacttgctcaccgcaaattcaaggtcgttatctgggatacccggatgtgaagctgaaatatccgagcagggtgcgcctgctacttctgctcctacaaacctccttgatgattatcctgcagcatctgggcgcaagtcttcttcaggaggctccccactatcctcctcaccccgttttagcgaccagcctgtgatgttagatgtttactcacctgatagattggccggagaactcttcttcttagacgtttcactaaaactcacagcagaagagttatcccgagctcctgcagaagttctaggtagaagcagccatggaacactgtacaaagcaacactggataatggacacatgttgacggtgaaatggttgagagttggcctcgtgaggcataagaaagatttcgcacgggaagccaaaaaaatcggctctttaaaacatcctaacattgttccgttgcgagcatactattggggtcccagagaacaagaaagacttctcctgtctgattacctgagaggagagagcttagccatgcatctttacgagactactcctcggaggtattctccaatgtccttcagccaaagactaaaagttgcagttgaagtggctcagtgtcttctctaccttcatgatagagcaatgccacacgggaacttgaagccaactaacataatcctctcaagtcctgacaataccgtgcgaataactgattactgtgttcaccgtttgatgactccatctggtgtagcggaacagatactaaacatgagtgcactcggttactctgctcctgaactttcctcggcttccaaaccgataccaacactcaaatccgatgtctatgcgtttggtgtgattctcatggagcttttgactagacggagcgcaggtgacataatctcaggtcaaacaggagccgttgatctgaccgattgggtgaggttgtgtgatcaagaaggaaggagaatggattgcatagacagagacatagcaggtggggaagagttctcaaagggaatggaggatgcacttgctgttgcaatcaggtgcattctctctgtcaatgaaaggcctaacatcagacaagttcttgatcatcttacttcaatttctgcctgagatttgtttgtttttttatctcttctaaatttttctgtaaatctccatggatttcctttcacacatcatcccattttttcttcttcttttctggttttatgtttctctctagtagtttgatcagatgattcagaatgatcctcaggtgttcttacatttattttttgttgattatttttccccttccttgtaaaggtagaagaaaatctaatcccattgattctgacagaaacatttgtgtttgcttattgattcttttttttataaattgttttttttatctcttttcgact

SEQ ID NO: 12 - protein, 1048 aa, Arabidopsis thaliana
MSHFLTFCFLSLLLLLHGANAVTETELRSLLEFRKGIRDETSHQRISWSDTSSLTDPSTCPNDWPGISCDPETGSIIAINLDRRGLSGELKFSTLSGLTRLRNLSLSGNSFSGRVVPSLGGISSLQHLDLSDNGFYGPIPGRISELWSLNHLNLSSNKFEGGFPSGFRNLQQLRSLDLHKNEIWGDVGEIFTELKNVEFVDLSCNRFNGGLSLPMENISSISNTLRHLNLSHNALNGKFFSEESIGSFKNLEIVDLENNQINGELPHFGSQPSLRILKLARNELFGLVPQELLQSSIPLLELDLSRNGFTGSISEINSSTLTMLNLSSNGLSGDLPSSFKSCSVIDLSGNTFSGDVSVVQKWEATPDVLDLSSNNLSGSLPNFTSAFSRLSVLSIRNNSVSGSLPSLWGDSQFSVIDLSSNKFSGFIPVSFFTFASLRSLNLSRNNLEGPIPFRGSRASELLVLNSYPQMELLDLSTNSLTGMLPGDIGTMEKIKVLNLANNKLSGELPSDLNKLSGLLFLDLSNNTFKGQIPNKLPSQMVGFNVSYNDLSGIIPEDLRSYPPSSFYPGNSKLSLPGRIPADSSGDLSLPGKKHHSKLSIRIAIIVASVGAAIMILFVLFAYHRTQLKDFHGRNRFTDQATTRDTKFGRSSRPSLFNFSSNVEQQSSSLSFSNDHLLTANSRSLSGIPGCEAEISEQGAPATSAPTNLLDDYPAASGRKSSSGGSPLSSSPRFSDQPVMLDVYSPDRLAGELFFLDVSLKLTAEELSRAPAEVLGRSSHGTLYKATLDNGHMLTVKWLRVGLVRHKKDFAREAKKIGSLKHPNIVPLRAYYWGPREQERLLLSDYLRGESLAMHLYETTPRRYSPMSFSQRLKVAVEVAQCLLYLHDRAMPHGNLKPTNIILSSPDNTVRITDYCVHRLMTPSGVAEQILNMSALGYSAPELSSASKPIPTLKSDVYAFGVILMELLTRRSAGDIISGQTGAVDLTDWVRLCDQEGRRMDCIDRDIAGGEEFSKGMEDALAVAIRCILSVNERPNIRQVLDHLTSISA

SEQ ID NO: 13 - DNA, 2367 bp, Solanum lycopersicum
atggataggtgggatacttcactgagtggattttacacctacccttttcgattcttgaatttatattcagaaattaaagatgggtctagctgggttcatgacaagaagaaggaaaaatggaagctttcttcttcttcttggaataatactctgttcttgtttgttgtgattgtgttttctgttttgcctgttatatcagctgggaggaattcagatggggttattgtgactcagtctgattttcaagctttaaaagctattaaacatgaattgattgattttaggggaatattgaagagctggaatgacactggtcttggagcttgtgctggtggttggctaggaattaagtgtgtcaatggggaagttatagctatacagttgccttggaagggattgggtggaagaatatctgaaaaaattgggcaattacaagctcttagaaagcttagtcttcatgacaatgttattgctggtcctgttccaacttctcttagcttcctaccaaatctcagaggtgtttatctgttcaataatcggctttcgggttcaatcccaccatcaattggaagaagccctcttcttcagactcttgatcttagcaacaatcagctcagtggtactatctctcctagtcttgcaagctccaccaggttgtacagactcaacttgagctacaatgcactttcagggtcaatcccagtaagtagtctcttaaggcttactgttcttgatttatctaataataccataaatggaactattcctgctagtttctcaaacctttcagctctttctactttggatttaaagagtaatcttttggatagccaaatcccagatactatgtatagaatgagaaacatgtcagtgctggatttgagcaacaacaaattcattggtcatattccagctactattggaaatatttctaggctcacctcacttgatttgtcgggaaacaatttcagtggtgaaattccagactctcttgtttctttggcaaatctgacttctttggatgtgtcttataacaatctttctgggattgttccatctcttctttctagaaagttcaattcaagtgcttttgttggaaatctagagctttgtggatatagtccctcaactccatgtgcttcaccaccccctcaaactgttccttctcctgttagtggggttgtcaagcctcaccgtcatcgtaaacttagtaccaaagatgtcattctcatagcatcaggtgctcttctagttgttctgcttcttttgtgctgtatgttactttgctgcttgattaggaaaaaggcaaattcgagagcgaaaaatggtggcaaagctggtggcctagctaccactacagggagaggtgcaaagtcagttcctgctgtaggaggggctgaagttgaatcaggtgaagctggtggaaagctagtccatttcgatggaccttttgtgtttgcagcagacgacttgttgtgtgccactgcagagatcatggggaagagcacttatgggacagcatataaggcaacattggaggatggtaatcaagttgcggtgaagaggctacgcgagaagatcacgaaagggcagaaagaatttgaagctgaggttgctgaattaggaaagattcgacacccaaatattttggcccttagagcctattacttgggaccaaaaggagaaaagcttcttgtgtatgattatatgtctaatggaagtctctcatccttccttcatgctagaggtcccgagacaacgattgactggccaacaaggatgaggattgctatcggtataacaaaaggaatttgctttctgcatacgaaagaaaacataattcatgggaatcttacatcaagcaacattttacttgatgagcataacaatccaaatattgcagatgttggactctccaagcttatgaccactgctggaaacaccaacgtaattgccactgcaggcacactaggatatcgcgcaccagagctttctaaaatcaagaatgcaagcaccaagactgatgtctacagtcttggagtgatcattttggagctcttaacaggaaaatccccaagcgaggcaacggatggtctcgatttgcctcagtgggtagcttccattgtgaaagaggagtggactaatgaagtgtttgatgtggaacttatgagggatgcacctaatattggtgatgagttgcttaacacattgaaactagctttacattgtgttgatcctacaccaactgctcgacccgaagctccgcaagtactacagaaattggaggagataaaaccagagatgatgttagcagctactagttctggagatgatggcacaacagttcaagaaaagagtgatgaataa

SEQ ID NO: 14 - protein, 788 aa, Solanum lycopersicum
MDRWDTSLSGFYTYPFRFLNLYSEIKDGSSWVHDKKKEKWKLSSSSWNNTLFLFVVIVFSVLPVISAGRNSDGVIVTQSDFQALKAIKHELIDFRGILKSWNDTGLGACAGGWLGIKCVNGEVIAIQLPWKGLGGRISEKIGQLQALRKLSLHDNVIAGPVPTSLSFLPNLRGVYLFNNRLSGSIPPSIGRSPLLQTLDLSNNQLSGTISPSLASSTRLYRLNLSYNALSGSIPVSSLLRLTVLDLSNNTINGTIPASFSNLSALSTLDLKSNLLDSQIPDTMYRMRNMSVLDLSNNKFIGHIPATIGNISRLTSLDLSGNNFSGEIPDSLVSLANLTSLDVSYNNLSGIVPSLLSRKFNSSAFVGNLELCGYSPSTPCASPPPQTVPSPVSGVVKPHRHRKLSTKDVILIASGALLVVLLLLCCMLLCCLIRKKANSRAKNGGKAGGLATTTGRGAKSVPAVGGAEVESGEAGGKLVHFDGPFVFAADDLLCATAEIMGKSTYGTAYKATLEDGNQVAVKRLREKITKGQKEFEAEVAELGKIRHPNILALRAYYLGPKGEKLLVYDYMSNGSLSSFLHARGPETTIDWPTRMRIAIGITKGICFLHTKENIIHGNLTSSNILLDEHNNPNIADVGLSKLMTTAGNTNVIATAGTLGYRAPELSKIKNASTKTDVYSLGVIILELLTGKSPSEATDGLDLPQWVASIVKEEWTNEVFDVELMRDAPNIGDELLNTLKLALHCVDPTPTARPEAPQVLQKLEEIKPEMMLAATSSGDDGTTVQEKSDE

SEQ ID NO: 15 - DNA, 2379 bp, Solanum lycopersicum
atggggtgtgttaaaaagataccttttgtccagattttgttgttcttgcagctgatgatttgtggagctcagttagctttaggtcaagtttgggatggtgtagttatcagtgaggcaaattatcaagcacttgaggcacttaaacatgagttagttgatcctaaagggtatttaagtagctggaatgatagtggttatggtgcttgttctggtacttggattgggataaaatgtgctcaaggacaagttattgttattcagcttccattaaaaggatttgggggaagaattactgaaagtattggtcaatttcaagcactaagaaaacttagttttcatgataatgaaattggtggttcagtcccttcatctttgggttttttacccaatctaagaggtttacaattgtacaataacaggttttcaggttctattcctggttctcttggtttatgtccagttctacaaacacttgaacttagcaacaattctttatctggtgcaatccctgatagtcttgtgaattcatctaagctttataggcttaatcttagttacaatttattgtctggttcgattcctgttagtataactcaatcgcgctctctagtctttattgacttgaaatataacaacttgacagggtctattccggattcttggggtggaaatggagatagacagtttaaattagagtcgttgacgcttgatcataactctttgtctggtggaattcctgtttcttttgggaagttgagtgaacttcttgagatttcgtttagtcataatcgtattgttggtgtcatccctaatgatattggaaggcttagtgtggttagggatcttgatttttcttataatgagattaatggtagtttgccagagagtataactaatttgtcctccctcatggttctcaacttggaaagtaacaatcttgatggtgaaatcccattagatgtaaagagattgcaaaagttgtcgtttctcaacctgaggaacaaccggtttaggggtgatattccagctgctatcggggacatttctggtctagtcgagatagatttgtctctcaacaatctaaccggagaaatcccagagtctgttagtgagttacctaatcttagttcctttaatgtttcatataactctctatctggtcctgttcctacttatcttgctaagaaattcaattcaagtgtttttgtgggtaatgttcagctatgtggctataatacttcgaatccatgtcctgtaacgccttctccagttacacgaagtagaaaacgaagtattaaagacattattctcgtagtaatagggtgtcttataatagtcttgtttctactctgttgcattctcctctgctgtttgatcaagaaaagaggtgaagccaaaaaaccaaaagatgtaaagggtgttcctccaactgccggagaagttgaggcagcaggaggggataacggaggaaaacttgtccattttggtggaggtatgatgttcagtgcagatgatcttctatgtgctactgcagatatattgggaaagagtacttatggtacggtgtatagggctacgttagaagatggaagtcaagttgcagtgaaaagaatgagagagaagatcacgaaagttcagagggaattcgagaaagaagtcaacgttcttggaaagataaggcatccgaatcttctggctattagggcttattacttagggacgaaaggggagaagcttcttattttcgactacatgcctaaaggaagtctctcaaattttcttcatgctcgtggaccggatacaccaattgattggccaacaaggatgagaatagcaaaagggacaacgaggggattgctgtaccttcatactaatgtcaacatcattcatgggaatcttacgtcgagcaatgttctacttgatgaaaacactgatgcaatagtttcagattatggtctttcacgccttgtaactgctgctgcaaattcaaatgttgtagccacagcaggtgcacttggttatcgcgcacctgaacttaataagctcaagaaagccaatgtgaagacggatgtctacagcctcggagtcatcatacttgaactcctaactggaaagtctccaggcgaggcagtgaatggtgtagatttgcctagaaaggtggcgtcaattgtgaaagaagagggaacaaaagaggtattcgatttggaactaatgaaggatgcatccataattggtgatgaattgttagttacacttaaattggctttgcactgtgttgatccctcgccattagctcgaccagaacttcagcaagttctccagcaactggaagacattaggcgtgaaacgccaactgcaggtccctcaacaagttactag

SEQ ID NO: 16 - protein, 792 aa, Solanum lycopersicum
MGCVKKIPFVQILLFLQLMICGAQLALGQVWDGVVISEANYQALEALKHELVDPKGYLSSWNDSGYGACSGTWIGIKCAQGQVIVIQLPLKGFGGRITESIGQFQALRKLSFHDNEIGGSVPSSLGFLPNLRGLQLYNNRFSGSIPGSLGLCPVLQTLELSNNSLSGAIPDSLVNSSKLYRLNLSYNLLSGSIPVSITQSRSLVFIDLKYNNLTGSIPDSWGGNGDRQFKLESLTLDHNSLSGGIPVSFGKLSELLEISFSHNRIVGVIPNDIGRLSVVRDLDFSYNEINGSLPESITNLSSLMVLNLESNNLDGEIPLDVKRLQKLSFLNLRNNRFRGDIPAAIGDISGLVEIDLSLNNLTGEIPESVSELPNLSSFNVSYNSLSGPVPTYLAKKFNSSVFVGNVQLCGYNTSNPCPVTPSPVTRSRKRSIKDIILVVIGCLIIVLFLLCCILLCCLIKKRGEAKKPKDVKGVPPTAGEVEAAGGDNGGKLVHFGGGMMFSADDLLCATADILGKSTYGTVYRATLEDGSQVAVKRMREKITKVQREFEKEVNVLGKIRHPNLLAIRAYYLGTKGEKLLIFDYMPKGSLSNFLHARGPDTPIDWPTRMRIAKGTTRGLLYLHTNVNIIHGNLTSSNVLLDENTDAIVSDYGLSRLVTAAANSNVVATAGALGYRAPELNKLKKANVKTDVYSLGVIILELLTGKSPGEAVNGVDLPRKVASIVKEEGTKEVFDLELMKDASIIGDELLVTLKLALHCVDPSPLARPELQQVLQQLEDIRRETPTAGPSTSY

SEQ ID NO: 17 - DNA, 2499 bp, Solanum lycopersicum
atgagagatcaagatggagtttttactagttttgaaccatacccttttcgagttaacaagtttttaactcatcttgcaaaatttgctcaacttttcttgttatttcagttactgatatttgggattcagccaacttcaggtgaagattgggatgggataattattactgcagcagattttcaagcacttcaagcttttaaacaagaattgattgatccaaaagggttcttgaaaagctggaatgatagtggaattggagcttgttctggtggatggcttggtattaaatgtgctcaaggacaagttattgttattcagcttccatggagaggtttaggtggaagaattactgaaaggattggacaatttcagtctcttagaaaactcagtttacatgataatgtcattagtggttcaattccttcaactttgggactcatacctaatcttagaggacttcaactttttaacaataggttatctggttctattcctgcttctcttggtttatgccctcttcttcaaacacttgatctcagcaataactcattctctggtgcaattccacctagtcttgttaattcaactaagctttataggcttaatcttagccataactctttgtctggttcaatccccacaagtctcgcacaatctccctcactcatctttcttcatctcaactataacaatctttcaggctcaataccagatacttgggatggaaatggaaaaagactcttccaacttcagtctcttacacttgatcacaacttcttttctggaagtattcctgcttccttaggcaagttgaatgaacttgttgagctttcacttagtcataatcaattgactggagttatcccaagtcattttgggggactctctagtcttacaacactggatttgtcttataatgctattaatggaagcttgcctgatagcttcttgaatctatcctcactagtggtcttgaacttggaaagcaaccaacttgataaccaaatcccagcagccataataaaattgcagaaactctcagttctcaacttgaggggcaaccattttagtggtgatattccagtcactataggaaacatctctactcttagacaactagatttagctcataacaatataagtggagaaatccctgcttctttagagaatttaccaaatcttagagccttcaatgtctcatataatgatctgtctggtcccgttccgactcatctcgcacggaaattcaactcgagtgcctttgtgggtaatcttcagctatgtggttacagtgcatcaaccccatgtccaatctctccagttagcccatcctccgaaacaccaaaaaggcaacgtcgtaagttaagtaccaaggacataattctcatagcaggaggggcacttctcataatcttggctctgctgtgttgcattctcctatgctgcttgatcaggaaaagatctgcggctagatcagggaaggatggacaaggcacgagtagggcggcaggtgaaaagggtgttccagcaactgctggagaagttgaggcagcaggaggaggagatacaggaggaaagcttgtccattttgatggacctattgtgttcacagcagatgatcttctttgtgcaaccgctgaaataatgggaaagagcacttatggtacggtgtacaaggccacattagaggatggcgatcaagttgcagtgaaaagattgagagagaagattacgagaggtcaaagggaatttgagagtgaagtcaatattcttgggaagatcaggcatccaaatcttcttgcccttagagcatattacatgggacctaaaggagagaaacttcttgtatttgactacatgcctaagggaagtttggcaacttttcttcatgctcgcagtcctgatacaccaattgattgggcaacaaggatgagaatagcaaagggtacaacaaggggattgctcttccttcatacaaatgccaatatcattcacgggaatcttacatcaagcaatgttttacttgatgacaacacaaatgcaaagattgcagattatggtctttcacgtcttatgactgctgcagcaaatgcaaatgtgattgcaactgctggagcacttgggtatcgagcacctgaactttcaaaactcaagaaagcaaacacaaagacagatgtatacagccttggtgttattatactggaacttctaactggcaagtcaccaggagaagcaatgaatggtgtggatttgcctcaatgggttgcatcaattgtgaaagaggagtggacaaatgaggtatttgatttagaactaatgagggatgcatccgttatcggggatgaattattgaacaccttgaaattggctttgcattgtgttgatccatcaccatcagctcgaccagaagttcagcaacttctccagcaactagaagaaattagacctgaaacagctaccagttctggggatgacggtgccgcagcaagcgatgattag

SEQ ID NO: 18 - protein, 832 aa, Solanum lycopersicum
MRDQDGVFTSFEPYPFRVNKFLTHLAKFAQLFLLFQLLIFGIQPTSGEDWDGIIITAADFQALQAFKQELIDPKGFLKSWNDSGIGACSGGWLGIKCAQGQVIVIQLPWRGLGGRITERIGQFQSLRKLSLHDNVISGSIPSTLGLIPNLRGLQLFNNRLSGSIPASLGLCPLLQTLDLSNNSFSGAIPPSLVNSTKLYRLNLSHNSLSGSIPTSLAQSPSLIFLHLNYNNLSGSIPDTWDGNGKRLFQLQSLTLDHNFFSGSIPASLGKLNELVELSLSHNQLTGVIPSHFGGLSSLTTLDLSYNAINGSLPDSFLNLSSLVVLNLESNQLDNQIPAAIIKLQKLSVLNLRGNHFSGDIPVTIGNISTLRQLDLAHNNISGEIPASLENLPNLRAFNVSYNDLSGPVPTHLARKFNSSAFVGNLQLCGYSASTPCPISPVSPSSETPKRQRRKLSTKDIILIAGGALLIILALLCCILLCCLIRKRSAARSGKDGQGTSRAAGEKGVPATAGEVEAAGGGDTGGKLVHFDGPIVFTADDLLCATAEIMGKSTYGTVYKATLEDGDQVAVKRLREKITRGQREFESEVNILGKIRHPNLLALRAYYMGPKGEKLLVFDYMPKGSLATFLHARSPDTPIDWATRMRIAKGTTRGLLFLHTNANIIHGNLTSSNVLLDDNTNAKIADYGLSRLMTAAANANVIATAGALGYRAPELSKLKKANTKTDVYSLGVIILELLTGKSPGEAMNGVDLPQWVASIVKEEWTNEVFDLELMRDASVIGDELLNTLKLALHCVDPSPSARPEVQQLLQQLEEIRPETATSSGDDGAAASDD

SEQ ID NO: 19 - DNA, 3156 bp, Solanum lycopersicum
atgaagttattcaagttcttgataatttttgttctttattttggctctgtcatcgggcaacttccctcccctgatatattggctttgcttgagtttaagaaaggtgttgaacatgatccaacagggtttcttcttgaatcttggaatgaggaatctattgatttcaatgggtgtccttcatcttggaatgggataatgtgtaatggtggtaatgttgcaggtgttgttcttgataacttgggtctagttgctcatgttgatttgagtgtatttgctaatcttacaaagcttgttaagctttcaatggctaacaattcaattgttgggaaaatgcctaataatataggtagattgaagagtcttcagtatctagatctctctggtaatttgttcaactcttctatacctccagaggttggagagttagggaaattaaagaatctgtcattagctggtaataacttctccggtatgataccggagacaatttcagggcttgtgtctatgcaatctttggacttgagtcacaattctttttctggggctctaccttcatctttgatgaagttggctggtttggtataccttaatctatctgttaatggattcacaaaggaaattactaaaggttttgaactgatggaaaatcttgaagtgctcgatttgcacggtaatatgtttgatggcaatttggatgtggaaatgttgatgcttaccactgcaactcatgttgacttgagtggaaatttgctagtgaacactgcttctcagcaggagaagtttttgccaggtttatcggagtcgattaagtatttaaatcttagtcataatcagctaaaaggttcacttgtaagtggaaatgaagctcagatatttggcaacttgaaggtcttggatttgagctacaatcagctctcaggggaattgcctagtttcaattttgtttatgatctacaggtgcttaaacttgccaacaacttattttccggatttattccgaatgatctgttgaaaggagatgcattgcttctcactgagttggatttaagtggaaacaatctcactggttcgataagcatgataacatccacaacattgcgaacacttaacttgtcctccaatgctctttctggtgaacttccctttgtgactggaacttctgcggtgcttgatctctcgaaaaatcagttcaaaggtaatctaactagaatgctgaaatgggggaatgttgaatttcttgaccttagccaaaaccatttgactggaaacatccctgaggttacggctcagtttctgcgtttatattacctcaacctttcaggcaatacgctcacaggatctctcccgaaagttatcacacagtttcctaagatcactgttcttgatctcagtttcaaccagttggatggggatcttcttacttctctactaactttgcccactattgaagaactccacctacaaaacaatgcatttgttggaagcattgatttccctcctccggctagtactcctaagcttcgtgttctagatctttgtcataaccagctcgctggttactttcctgacggatttggatcattgattgcacttcaagtactcgatatatcaggaaataatttctctggatctttgccaacttccatgggtaatgttagctctcttacttctttagatgtttcagagaatcatttctcgggtgaactgccaaaaaaccttcctaacagtatccaaagctttaatgcatcactcaatgacttctccggtgtcgtacctgagaatttgagaaagtttcctttatcatctttctacccgggaaactctcaacttcagtttccaaatcctccctcaggatcgggtcaagcttcagcagaaaagcataagagcaggcaacttaaaactattatcaaagtggtgatcattgttgtttgtgtggttgttcttgttgttttaatcttgctagccatttttgtattctacttacgggcatcgaggaactctcaggcacatgttactgacaaagacatccgccgtcaaacccaatcaaatccttctggttttagcagtagagaaggtgcagttgtttcagcacaaaatgttgcagctgcttcaaggaaagcatcatcatcagaagtaattagtgctgatgagaaaacgacaggtttctccccttcgaaaacgagtcatttcacttggtcaccagagtcgggggaatcatatagtgcagaaagccttgctgataatttggctggagagctgttcttccttgatgatacaatctcttttacagctgaggaattatctagggccccagctgaagtcttaggcagaagcagccatggaacatcttacagggcaacactagataacggattgctcttgactgtgaaatggttgagagaaggagtggccaaacagagaaaagatttcacaaaggaggctaaaaagtttgccaacattaggcatccaaatgtggtgggattaagaggttactactggggtcctacacaacatgagaagcttattctttcagattacatatctcccggaagtctgtcaagctttctatatgatcggcctggaagaaaaggtccaccactaacctggccccaaagaatcaaaatatcagttgatattgcgcgtggcctgaattatctccattttgaccgtgaagttcctcatggaaaccttaaagcaaccaacgtcttgttggatggtcctgatcttaatgcacgagttggtgattactgccttcaccgtcttatgactcaagcggggacaattgaacaaattcttgatgctggagtgttaggctatcgtgcaccagagttggctgcatcaaagaaaccagtgccttccttcaaatcagatgtatatgctttcggagtcattctgttggaggtcttaactgggaaatgtgcaggtgatgttgtttccggtgaagatggtggtgtagacttgactgattgggtaaggttgaaagtggcagaagttggaggattcgactgttttgataatgcattgtcatctgatatgggaaatcaagcaatggagaagcaaatgaaggaggttcttgcaatagctctaagatgcatacgttcggtatctgagagaccaggtatcaagactgtatatgaagacctttcatctatttag

SEQ ID NO: 20 - protein, 1051 aa, Solanum lycopersicum
MKLFKFLIIFVLYFGSVIGQLPSPDILALLEFKKGVEHDPTGFLLESWNEESIDFNGCPSSWNGIMCNGGNVAGVVLDNLGLVAHVDLSVFANLTKLVKLSMANNSIVGKMPNNIGRLKSLQYLDLSGNLFNSSIPPEVGELGKLKNLSLAGNNFSGMIPETISGLVSMQSLDLSHNSFSGALPSSLMKLAGLVYLNLSVNGFTKEITKGFELMENLEVLDLHGNMFDGNLDVEMLMLTTATHVDLSGNLLVNTASQQEKFLPGLSESIKYLNLSHNQLKGSLVSGNEAQIFGNLKVLDLSYNQLSGELPSFNFVYDLQVLKLANNLFSGFIPNDLLKGDALLLTELDLSGNNLTGSISMITSTTLRTLNLSSNALSGELPFVTGTSAVLDLSKNQFKGNLTRMLKWGNVEFLDLSQNHLTGNIPEVTAQFLRLYYLNLSGNTLTGSLPKVITQFPKITVLDLSFNQLDGDLLTSLLTLPTIEELHLQNNAFVGSIDFPPPASTPKLRVLDLCHNQLAGYFPDGFGSLIALQVLDISGNNFSGSLPTSMGNVSSLTSLDVSENHFSGELPKNLPNSIQSFNASLNDFSGVVPENLRKFPLSSFYPGNSQLQFPNPPSGSGQASAEKHKSRQLKTIIKVVIIVVCVVVLVVLILLAIFVFYLRASRNSQAHVTDKDIRRQTQSNPSGFSSREGAVVSAQNVAAASRKASSSEVISADEKTTGFSPSKTSHFTWSPESGESYSAESLADNLAGELFFLDDTISFTAEELSRAPAEVLGRSSHGTSYRATLDNGLLLTVKWLREGVAKQRKDFTKEAKKFANIRHPNVVGLRGYYWGPTQHEKLILSDYISPGSLSSFLYDRPGRKGPPLTWPQRIKISVDIARGLNYLHFDREVPHGNLKATNVLLDGPDLNARVGDYCLHRLMTQAGTIEQILDAGVLGYRAPELAASKKPVPSFKSDVYAFGVILLEVLTGKCAGDVVSGEDGGVDLTDWVRLKVAEVGGFDCFDNALSSDMGNQAMEKQMKEVLAIALRCIRSVSERPGIKTVYEDLSSI

SEQ ID NO: 21 - DNA, 3075 bp, Solanum lycopersicum
atggggcagcttccttcccaggacattttggccctgcttgaatttaggaagggtataaaccatgatccaactggttatgtactgcagtcatggaatgaagagtccattgatttcaatggttgcccttcatcttggaatggtataatgtgcaatggtggtaatgttgcagcagttgtccttgataacatgggtttatctgctgatgcagatttgagtgtgtttgctaacctcacaatgcttgtgaaactctctatggctaacaattcgattactggacaaatgccaaagaaaattggtgattttaagagtcttgaatatctggatatttcaaacaatctattcaactcctctttaccaccagagattgggaaaataggaagcttaaagaatctgtcactagctggtaacaacttctctggcccaatccctgatactatttcagagctcatgtcaattcaatctttggatttgagtcacaattctctttctgggctacttccttcttctttgaccaagttaaataatttagtatacctcaatctttctcttaatggatttacaaagaaagtcccaaaaggttttgagctaatggctaaccttgaagttcttgacttgcatggaaatatgcttgatggtactttggatccagagttcttactgcttactactgcaacttatgttgacttaagtggaaatttgctcgtgagttctgcttcgcaacatgagaaatttttaccaggcatatcctcgtcagtcaagtacttgagtcttagccataatcagcttacggggtcacttgtaagtggtggtgaagctcaggcatttggaaacttgaaggttctggatttgagctataatcagctgtccggggaattacctggctttaattttgtttatgatcttcaggtcctgagactcagcaacaacagattttctggtttcgttccaaatgatctcttgaaaggagatgctttagtcctctcagaattggatttaagtggaaacaacctcacagggtcgataagcatgattacatcaacaactctgcgtgttcttaacttatcctccaatgctctttctggtgaacttccactggtgacaggtagtactgcagttcttgatctctctaaaaaccaattagaggggaatctaactagaattcagaaatgggggaatgttgaatttcttgacctcagccagaaccaattgactggaaacatccctgaggttacagctcagtttctgcgattaaatcgcctaaacctttctcacaatgctcttactggatctatcccaaaagttatcacacagtttcccaagatcacagtccttgatcttagtttcaaccagctgaatggacctcttctcacttctctgctaacagtacccactattgaagagcttcaccttcaaaacaatgcacttgttggaaatattgatgtcgctgccccctctgctacacccaacctccgtgttcttgatctttctcataatcagcttgctggctcttttcctgatgggtttggtttgttgactgcgcttcaagtacttgatatagctgacatttcacagaatcattttactggtcctttgccgatgaacctgcctgatggcctccaaagcttcaatgcatcactcaatgacttatctggtgttgtccctgataatttgagaaagttccctttatcagctttctaccctggaaactctgaacttcaatttccaaatcctccctcaggatctggccaagcttcaccagaaaaccaaaaaagtcgatcacttaaaactatcatcaagttggtaataatagtttcttgtgtaattgcttttattattttggtcctgctagtcattttcttttattatatacgagcttcaaggaagcgtcatcctcgtgtaactgaaaaagtagttcatcgccaagccacatcaaatccttctggctttagcagtagggagggtgccggtggtgcagttgtttccgctgaagatcttatgacttcacggaaagggtcatcagaaataattagcccagatgagaaaatggctgctataactggtttctccccttcaaaaggtagccatttctcctggtcaccagagtctggagattcgtatactgcagaaacttttgcaagattggatgtgaagtctccggatcgtctggctggagagctgtacttccttgatgatacaatctcttttacacctgaggaactttccagggctccggctgaagtcttaggcagaagcagccatggtacatcatacagggcaacactggagaatgggttgcttctgactgtgaaatggttgagagaaggagtggcgaagcagaggaaggattttgcgaaggaggctaaagagtttactaatattaggcatcctaatgtagtaggattaagaggttactactggggtcccacacaacatgagaagctcattctttcggattatatatctcctggaagtcttgcaagtttcctctatgatcggcctggtagaaaaggtccaccactaacctggccccaaagactcaaaatatcagttgatgttgcacgtggcttgaactatctccattttgatcgggaggttccacatggaaatcttaaagcaaccaacatcttgttggatgggcctgaccttaatgcaagggttgcagattactgccttcaccgcctcatgactcaagccggcacaatagaacagattcttgatgcaggagttttgggttatcgtgcccctgagttggctgcatcaaagaaaccactgccttccttcaaatcagatgtatatgcttttggagttattttgttggagctgcttagtggaaaatgtgcaggtgatgttgtctctggcgaagatggcggagtagacttgactgactgggtaaggttgaaggtggcagaaggtcgtagctctgattgttttgataacgttttgtcacctgagttggaaaatccagcaatggagaagcaaatgaaggaggttcttggtatagctgtgcgatgcattcgttctatatctgagagacctggtatcaagactatatatgaggacctttcgtctatatag

SEQ ID NO: 22 - protein, 1024 aa, Solanum lycopersicum
MGQLPSQDILALLEFRKGINHDPTGYVLQSWNEESIDFNGCPSSWNGIMCNGGNVAAVVLDNMGLSADADLSVFANLTMLVKLSMANNSITGQMPKKIGDFKSLEYLDISNNLFNSSLPPEIGKIGSLKNLSLAGNNFSGPIPDTISELMSIQSLDLSHNSLSGLLPSSLTKLNNLVYLNLSLNGFTKKVPKGFELMANLEVLDLHGNMLDGTLDPEFLLLTTATYVDLSGNLLVSSASQHEKFLPGISSSVKYLSLSHNQLTGSLVSGGEAQAFGNLKVLDLSYNQLSGELPGFNFVYDLQVLRLSNNRFSGFVPNDLLKGDALVLSELDLSGNNLTGSISMITSTTLRVLNLSSNALSGELPLVTGSTAVLDLSKNQLEGNLTRIQKWGNVEFLDLSQNQLTGNIPEVTAQFLRLNRLNLSHNALTGSIPKVITQFPKITVLDLSFNQLNGPLLTSLLTVPTIEELHLQNNALVGNIDVAAPSATPNLRVLDLSHNQLAGSFPDGFGLLTALQVLDIADISQNHFTGPLPMNLPDGLQSFNASLNDLSGVVPDNLRKFPLSAFYPGNSELQFPNPPSGSGQASPENQKSRSLKTIIKLVIIVSCVIAFIILVLLVIFFYYIRASRKRHPRVTEKVVHRQATSNPSGFSSREGAGGAVVSAEDLMTSRKGSSEIISPDEKMAAITGFSPSKGSHFSWSPESGDSYTAETFARLDVKSPDRLAGELYFLDDTISFTPEELSRAPAEVLGRSSHGTSYRATLENGLLLTVKWLREGVAKQRKDFAKEAKEFTNIRHPNVVGLRGYYWGPTQHEKLILSDYISPGSLASFLYDRPGRKGPPLTWPQRLKISVDVARGLNYLHFDREVPHGNLKATNILLDGPDLNARVADYCLHRLMTQAGTIEQILDAGVLGYRAPELAASKKPLPSFKSDVYAFGVILLELLSGKCAGDVVSGEDGGVDLTDWVRLKVAEGRSSDCFDNVLSPELENPAMEKQMKEVLGIAVRCIRSISERPGIKTIYEDLSSI

SEQ ID NO: 23 - DNA, 3177 bp, Solanum lycopersicum
atgactctctctcaatctctctctttactcctccttgtacttgtgtttcggctcaatcggtcttctgctgcagaagatgaagttcgatcacttttggagttcaaaaagggtataaaaaatgacccattaggtaaaatcttcagttcgtggagtcaaacaggtctttcagatccatcagcatgtccaaagtcgttttatggtgtcgtttgtgatgaaaactccgattctgttttctccatttctcttgacgggcttgggcttgtgggtgacttgaagttctcaactcttagtgggttgaagcagctcaaaattcttagtctttctggtaattcttttaccggtagggttgttcctgctttgggttctatgttaactcttcagcgtttggatctgtctgggaatcagttttatggaccaattccagcacgaattaatgagctttgggatttgaattacctcaatttgtccaataataattttacatttggttacccaagtggaattagtaatcttcagcagttgagagtactggatttgcataacaatgagctttggggtgatattggggagttgttcttggagttgaagcgtattgagtatcttgatttgagtaacaactcgttttttgggtctcttcctacgagcccggaaaatgtttcactcgcttcgacaattcatgtcatgaatttgagtcacaacaacttgggtggtggtttttttcctggaaaattgttggaggcatttgagaatttgatggttttggatttggggaataatgctataatggggcagcttccatctactggctttatgcataatttgagggttttaaggcttgggaataatcagctttttggattgattcctgatgagcttttgcagggaacaagtccattggtagaactagatcttagtggcaatggattttcaggttcaattccgatagtgaattcaacaaagttgagagttctaaatatttcatcgaaccacctattaggttcactgccatcttccataggaagttgtgcagttgtggatttgagcagaaatatgctcgtagatggtatatcagctaatgagagctgggaagccaatttggaaattattgatttgagttcaaataggttaactggaaatattcccaatatgacctgtcaatttcaactattgacttcacttaatttcggaaacaattctcttgagggaaccttaccttcatcactgggcaccttgccaagactggttaaacttgatctcagtaccaataaactcggtggactgattccatctacattttttacttcgacgacattgatgaacctaaatatatctggaaaccagttgtcaggatcaattcctcttgaaggcactcatgctagcgaattacttgttcaatcatcataccctacacttgagtctcttgatctttcagaaaacaccttgacaggtaatttgtcttctgccattggtaatctgcggaggcttcaagtgctaaatcttgctaagaatcagttatcaggcatgctacctactgaattgggtgaccttagaaatttggaattccttgatatatctaacaacaattttagtggtgtgatccccgaaaatctttcatcaaatttgagggtgtttaatgtgtctaacaatgaattaagtggtgctatcccggataacttgagaaactttaatgaaagttcatttcgtcctggaaactcaaatttggcaatcccaagcaactggttacatgataatcatggcaatcctggtcaaaatagtcagcaccatcataactcgaaatccagtatcagggtggctattattcttgcctcagttggagctgctttaatgattggtgttgttctcctggcttaccttcgacaacggttccaaaattttcacttgccaagtggatttaacagccaatctgctggaagggatgttaaactcgggaggttcagccggcctggaattttaaagttccatggctcctcagaaccaccaccaactttcttgagtttctctaatgatcacttgctcacagtgaactcgagatctctgtctggccaaattgagtctggtacagaaattgttgaacatgtctttctggagggagttactgcagtttcagcatctacgcatcttggcactgtgggtaataatcctgcaacatctggccggagatcttctccagactctccaatagctcactcccctcgtttcattgacacagttgaacaaccggtgacattggatgtgtgttcaccagatcgattggctggggaattattcttcctggatggttcactgtcatttactgctgaggagttatctcatgcccctgctgaagttctaggtagaagtagccatggcacattgtataaagctactttgaatagtgggtacattcttactgtcaagtggttgcgggttgggttggtaaaaatcaagaaagcatttgcaaaggaagttaaaaagattggatctatcaaacacccaaatgttgttcatttacgagcatattattggggacctcgagaacaagagaggcttattttagcagactacatttcaggagatagtttggcgttacatctctatgagacaacccctcggaggtactccccgttatccttcaaccaaaggttaaaagttgccattgaagttgctcagggtttggcatatctgcatgagagaggactgcctcatggcgacttgaaacctacaaatataatcttggttggtgctgattacagtgtccgcctcactgattatggtctccaccgcgtgatgactccagcagggattgtagagcagatactgagcctaggggctctaggataccgtgctcctgaacttgctactgctaccaaacctatcccatcatttaaggctgatgtatatgctcttggtgtgattctaatggaattgctgacgagaagaagtgctggagacataatctcatggcactctgctgcagttgatttaatagattgggttcggttgtgtgataaagaaggtaggggaatggactgtatcgatagggccattgcaggaggagaagaacactgcaaagcgatggatgatctgctagccgtgtcgttaaagtgtattcttccgataaacgaaaggccaaacatcagacaagttgttgaagatctgggttccatatctgtgtga

SEQ ID NO: 24 - protein, 1058 aa, Solanum lycopersicum
MTLSQSLSLLLLVLVFRLNRSSAAEDEVRSLLEFKKGIKNDPLGKIFSSWSQTGLSDPSACPKSFYGVVCDENSDSVFSISLDGLGLVGDLKFSTLSGLKQLKILSLSGNSFTGRVVPALGSMLTLQRLDLSGNQFYGPIPARINELWDLNYLNLSNNNFTFGYPSGISNLQQLRVLDLHNNELWGDIGELFLELKRIEYLDLSNNSFFGSLPTSPENVSLASTIHVMNLSHNNLGGGFFPGKLLEAFENLMVLDLGNNAIMGQLPSTGFMHNLRVLRLGNNQLFGLIPDELLQGTSPLVELDLSGNGFSGSIPIVNSTKLRVLNISSNHLLGSLPSSIGSCAVVDLSRNMLVDGISANESWEANLEIIDLSSNRLTGNIPNMTCQFQLLTSLNFGNNSLEGTLPSSLGTLPRLVKLDLSTNKLGGLIPSTFFTSTTLMNLNISGNQLSGSIPLEGTHASELLVQSSYPTLESLDLSENTLTGNLSSAIGNLRRLQVLNLAKNQLSGMLPTELGDLRNLEFLDISNNNFSGVIPENLSSNLRVFNVSNNELSGAIPDNLRNFNESSFRPGNSNLAIPSNWLHDNHGNPGQNSQHHHNSKSSIRVAIILASVGAALMIGVVLLAYLRQRFQNFHLPSGFNSQSAGRDVKLGRFSRPGILKFHGSSEPPPTFLSFSNDHLLTVNSRSLSGQIESGTEIVEHVFLEGVTAVSASTHLGTVGNNPATSGRRSSPDSPIAHSPRFIDTVEQPVTLDVCSPDRLAGELFFLDGSLSFTAEELSHAPAEVLGRSSHGTLYKATLNSGYILTVKWLRVGLVKIKKAFAKEVKKIGSIKHPNVVHLRAYYWGPREQERLILADYISGDSLALHLYETTPRRYSPLSFNQRLKVAIEVAQGLAYLHERGLPHGDLKPTNIILVGADYSVRLTDYGLHRVMTPAGIVEQILSLGALGYRAPELATATKPIPSFKADVYALGVILMELLTRRSAGDIISWHSAAVDLIDWVRLCDKEGRGMDCIDRAIAGGEEHCKAMDDLLAVSLKCILPINERPNIRQVVEDLGSISV

SEQ ID NO: 25 - DNA, 2496 bp, Capsicum annuum
atggataggtcaatgagtcgaatcagagatcatagtcttttgttggttcttgtaataatagtgttttccatttctccgtttgtatcatctagtcggagtagaacggatggggtcattgtgaagcagtctgattttcaagctctacgagcgataaagaatgaactgattgattttcgaggaatattgaagagctggaacgacagtggtctcggggcttgcggtggaggttggttgggaatcaagtgtgtgaacggggaagtcatcgcgatacagttaccatggaaaggattgggtggcagaatatccgagaagataggacaattgcgagcgcttagaaagattagtcttcacgataatgtgattggtggtgttgttccaacttctttaatcttgttaccaaatttaagaggtgtttatctgttcaataatcgactttcgggttcgattccaccatcaattggaagaagaagtccagttcttcagactcttgatcttagcaacaaccaactcactggtactgtctctccaaatcttgcgaattccgccaggttatacagactcaacttgagctacaatgcactttcggggtctatcccagtaagtttaactcgatcatcttctcttacttttctcgcacttgaacataataatctttctggttttattcctgatacgtggggcggtatggttagcatgaataagtcgtaccaacttcagtatcttacacttgatcataatcgtctatctgggaaaattccagcttcgattagtaagttaagtatgctcgaggagattaatctgagtgataatcgtattaatgggaccattcctgatgaattaggcggtctctcgaggcttactgttctcgatttgtctaataatctcatagatggagctattcctgctagtttttcgaacctttcagctcttggtactttggttttaaagagtaatctactcgataaccaaatcccggaggacattgatagattgaaaaacctttcaatgctggatttgagtaacaacaaattcgttggtcgtattccagctactattggaaatatttccggacttgcctcacttgacttatctgaaaacaatttcaccggtgaaattccaaactctcttgtttcgttggaaaatctgacttcgttggatgtctcttataacaatctttccgggattgtcccatctcttctttctaaaatgttcaattcaagtgcttttgttggaaatctagaactttgtggatatagtccctcaactcaatgtgcttcaccgccccctcagactcttccttctcctgttagcggggttgtgaagcctcgccatcgcaaacttagtaccaaggatatcattctcatagtatcaggagctcttctagttgttctacttcttttgtgctgcatgttactttgctgcttgattaggaaaaaggcaaattcgaaagcgaaaaatggtagcaaagccagtggcctagctactaccgcagggagaggtgcaaaggcggttccggaagtaggagggactgaagttgaatcaggtgaagctggtggaaagctagtccattttgatggacctttcgtgtttgcagcagacgacttgttatgtgccactgcagagatcatggggaagagcacttatgggacagcatataaggcaacattggaggatggtaatcaagttgctgtgaagaggctacgcgagaagatcacaaaagggcagaaagaatttgaagctgaggttgctgaattaggaaagattcgacacccaaatattttggcgcttagagcgtattacttgggaccaaaaggagaaaagcttcttgtctatgattatatgcctaatggaagtctctcatccttcctccatgctagaggtcccgagacaacgatagactggcctacaaggatgaggattgctatcggtataacaaaaggaatctgctttctgcataccaaagaaaacataatacatgggaatcttacgtcaagcaacatactgcttgatgaacaaaacaacccaaagattgcggatgtgggactctccaggcttatgactactgctggaaacaccaacgtgattgccactgcaggcacgctaggttatcgtgcaccggaactttccaaaatcaagaatgcaagtaccaagactgatgtctatagtcttggagtgatcattttggagctcttaactggaaaatcccctagcgaggctacggatggtctcgatttgccacagtgggtagcttccattgtgaaagaggagtggactaatgaagtgtttgatgtggaacttatgagggatgcacctaatattggtgatgagttgcttaatacattgaaactggctttgcactgtgttgatcctacaccaactgctcggccagaagctccgcaagtactacagaaattggaggagataaaaccagagctgatgttagcagccaccagttctggagatgatggcacagcagttcaagaaaagagtgaataa

SEQ ID NO: 26 - protein, 831 aa, Capsicum annuum
MDRSMSRIRDHSLLLVLVIIVFSISPFVSSSRSRTDGVIVKQSDFQALRAIKNELIDFRGILKSWNDSGLGACGGGWLGIKCVNGEVIAIQLPWKGLGGRISEKIGQLRALRKISLHDNVIGGVVPTSLILLPNLRGVYLFNNRLSGSIPPSIGRRSPVLQTLDLSNNQLTGTVSPNLANSARLYRLNLSYNALSGSIPVSLTRSSSLTFLALEHNNLSGFIPDTWGGMVSMNKSYQLQYLTLDHNRLSGKIPASISKLSMLEEINLSDNRINGTIPDELGGLSRLTVLDLSNNLIDGAIPASFSNLSALGTLVLKSNLLDNQIPEDIDRLKNLSMLDLSNNKFVGRIPATIGNISGLASLDLSENNFTGEIPNSLVSLENLTSLDVSYNNLSGIVPSLLSKMFNSSAFVGNLELCGYSPSTQCASPPPQTLPSPVSGVVKPRHRKLSTKDIILIVSGALLVVLLLLCCMLLCCLIRKKANSKAKNGSKASGLATTAGRGAKAVPEVGGTEVESGEAGGKLVHFDGPFVFAADDLLCATAEIMGKSTYGTAYKATLEDGNQVAVKRLREKITKGQKEFEAEVAELGKIRHPNILALRAYYLGPKGEKLLVYDYMPNGSLSSFLHARGPETTIDWPTRMRIAIGITKGICFLHTKENIIHGNLTSSNILLDEQNNPKIADVGLSRLMTTAGNTNVIATAGTLGYRAPELSKIKNASTKTDVYSLGVIILELLTGKSPSEATDGLDLPQWVASIVKEEWTNEVFDVELMRDAPNIGDELLNTLKLALHCVDPTPTARPEAPQVLQKLEEIKPELMLAATSSGDDGTAVQEKSE

SEQ ID NO: 27 - DNA, 2406 bp, Capsicum annuum
atggaggaccaagaaagagttgaaagactcattatttcaaacgaaaaatggatatcatttatccaaattttcttgttcttgcaactactaatttctagtactcggttaactttatgccaagactgggatggtatagttatcactgaagcaaattatcaagcccttgaagcacttaaacatgagttagttgatccacaagggtacttgagtggttggaatgacagtggatatggtgcatgttcaggtacttgggtgggaattaaatgtgctaaaggacaagttattgttataatgcttccattgaaaggcctaggtggtagaataagtgaaagtattggtcagttacaagaactcagaaaactgagtttacatgataatgagataactggttcagtgcctttatcattgggttatttacctaatcttagaggtgttcagctttacaataacaggttatctggtactattcctagttcacttggtttatgtccagttctacaaacacttgaacttagtaacaatatgttatctggtgtaatacctgatagtcttgttaattcaactaagctatataggcttaatcttagttataattcattgtctggtagtatacctgcaagtgtcactcaatcgcgctctttagtctttattgacctgagatataacaatttgtcaggttctattcctgattcttggggtggaaatggagagagagttagattacagtctttggtactcgatcacaactcgtttactggaggaattcctgtttcttttggcaagttgaatgagcttcttgagatttcacttagtcataatcatattgttggtgtcattccaaatgatattggaaggcttagtatggttagaaatcttgatttttcctgcaatgagattaatggaagtttgcccgaaagcctatccaatttgtcctcccttgtagctctcaacttggagagcaacaatcttgatggtgaaatcccattagatataaacaaattgcaaaagttgttgattcttaacctgaggaataaccggtttagaggtgatattccagctatcattggggacatttctggtcttgtcgagatagatttatctcgtaacaatttgagtggagaaatcccagtgtctgttggtgatttcccaaatcttagttcctttaatgtttcatataactctctgtctggtcctgttcctacttatcttgctaagaagttcaattcaagtgcctttgtgggtaatgttcagctatgtggctataatacttcgaatccatgtcctggtacgccttcaaaggctacacgaggtagcaaacgaagtattaaggacattattctcatagtaataggtgctcttataataattttatttctagtctgttgcattctcctctgctgcttgatcaagaaaagaaatgaagccaaaacagctaaagatgtaaaaggtgttcctccaactgccggagaagttgaagcggctggagatactggcggaaaacttgtccattttggtggaggtatgatattcagtgcagatgatcttctatgcgctactgcagatatattgggaaagagcacttacggtacggtgtataaggccacattagaagatggaaatcaagttgcagtgaaaagaatgagagaaaagatcacaaaagctcaaagggaatttgagaaagaagtcaatgttcttggaaagataagacatcctaatcttctggctattagagcttattacatagggacaaaaggggagaaacttcttattttcgactacatgactaaaggaagtctagcaacttttcttcatgctcgtggccccgatacaccagtggattggccaacaaggatgagaatagcaaaagggacgacgaggggattgctgtaccttcacactaatgccaatatcattcatggcaatcttacgtcgagcaatgttctgcttgatgaaaactcagatgcaaaaattgcagattatggcctttcacgccttgttactgctgctgcaaatacaaatgtaattgccacagccggggcacttggctatcgtgcacccgaacttaataagctgaagaaagccaatgtgaagacggatgtctacagccttggagtcattatattggaactcctaactggcaagtctccgggcgaagtaatgaatggtgttgatttgccaagaaaggtggcgtcaattgtgaaacaggagggaacaaatgaggtgtttgatttggaactgatgagggatgcatccataattggcgatgaattgttaattacactgaagttggctttgcactgtgttgatccctcaccatcagctcggcccgaacttcggcaagttctccagcaactggaagacattaggcgtgatacgccgactgcagctccctcagcaagtcactag

SEQ ID NO: 28 - protein, 801 aa, Capsicum annuum
MEDQERVERLIISNEKWISFIQIFLFLQLLISSTRLTLCQDWDGIVITEANYQALEALKHELVDPQGYLSGWNDSGYGACSGTWVGIKCAKGQVIVIMLPLKGLGGRISESIGQLQELRKLSLHDNEITGSVPLSLGYLPNLRGVQLYNNRLSGTIPSSLGLCPVLQTLELSNNMLSGVIPDSLVNSTKLYRLNLSYNSLSGSIPASVTQSRSLVFIDLRYNNLSGSIPDSWGGNGERVRLQSLVLDHNSFTGGIPVSFGKLNELLEISLSHNHIVGVIPNDIGRLSMVRNLDFSCNEINGSLPESLSNLSSLVALNLESNNLDGEIPLDINKLQKLLILNLRNNRFRGDIPAIIGDISGLVEIDLSRNNLSGEIPVSVGDFPNLSSFNVSYNSLSGPVPTYLAKKFNSSAFVGNVQLCGYNTSNPCPGTPSKATRGSKRSIKDIILIVIGALIIILFLVCCILLCCLIKKRNEAKTAKDVKGVPPTAGEVEAAGDTGGKLVHFGGGMIFSADDLLCATADILGKSTYGTVYKATLEDGNQVAVKRMREKITKAQREFEKEVNVLGKIRHPNLLAIRAYYIGTKGEKLLIFDYMTKGSLATFLHARGPDTPVDWPTRMRIAKGTTRGLLYLHTNANIIHGNLTSSNVLLDENSDAKIADYGLSRLVTAAANTNVIATAGALGYRAPELNKLKKANVKTDVYSLGVIILELLTGKSPGEVMNGVDLPRKVASIVKQEGTNEVFDLELMRDASIIGDELLITLKLALHCVDPSPSARPELRQVLQQLEDIRRDTPTAAPSASH

SEQ ID NO: 29 - DNA, 918 bp, Capsicum annuum
atggttgctataactagtttctccccttcaaaaggtagccatttctcttggtcgccagagtccggagaatcgtatgctgcagaaaattttgcaagattggatgtgaggtatccagatcgtctggatggagaactgtatttcgttgatgatacaatcgcatttacacctgcggaactttccagggctccggctgaagtcctaggtagaaacagccatggtacatcttacagggcaacactagagaatgggttgctcttgactgtgaaatggttgagagaaggagtggcgaagcagagaaaggattttgcgaaggaggctaaaaattttgccaatattatgcatcctaatgtagtaggattaagaggttactactggggtcctccacagactgagaagctcattctttcggattatgtatctcctggaagtcatgcaagtttcctctatgatcgacctggtaaaaaaggtccaccactagcctggtctcaaagactcaaaatatcagttgatgttgaacgtggcttgaaccacctccattttgaccgtgaggttccacatggaaaccttaaagcaaccaacatcttgttggatgggcctgaccttgatgcaagagttgcggattactgccttcaccgtctcacgacacaagccggcacaatagaacagattctggatgcaggagtgttgggttatcgtgcccctgagttggctgcgtcgaagaaaccactgccttccttcaaatcagacgtatatgcttttggagttattttgttggtgctgctattgtcaatgccaatatgtatggtgctgactcgggcatccttgcgtgtacaggtgagcaaatttgaaggtctcagggtatttaataatgaggctggtgaaagtattattattcagttatcattgacagtacaataa

SEQ ID NO: 30 - protein, 305 aa, Capsicum annuum
MVAITSFSPSKGSHFSWSPESGESYAAENFARLDVRYPDRLDGELYFVDDTIAFTPAELSRAPAEVLGRNSHGTSYRATLENGLLLTVKWLREGVAKQRKDFAKEAKNFANIMHPNVVGLRGYYWGPPQTEKLILSDYVSPGSHASFLYDRPGKKGPPLAWSQRLKISVDVERGLNHLHFDREVPHGNLKATNILLDGPDLDARVADYCLHRLTTQAGTIEQILDAGVLGYRAPELAASKKPLPSFKSDVYAFGVILLVLLLSMPICMVLTRASLRVQVSKFEGLRVFNNEAGESIIIQLSLTVQ

SEQ ID NO: 31 - DNA, 3180 bp, Capsicum annuum
atgactcctcctcaatctctctctttactcctcctggcacttgtgttcatgctcaatggttgttcttcagcagtagaagatgaaattcgatcacttttggagttcaaaaaaggtattaaaaacgacccattaggcaaaatcttcagttcatggataccaagtccttcagatctatcagcgtgtcccagttcattctatggcgttgaatgcgatgccaactccaattctgttgcttccattacccttgacggacttggacttgtaggtgacttgaagttctcaactctaaatgggttgaaacagcttaaagttcttagtctttcgggtaactcttttacgggccggattgttcctgctttgggcacgttggtcactcttcagcatttggatctgtcggggaatttgttttatgggccgattccagctagaattaatgagctttggggtttgaattatcttaatttatcaaataataattttaatgggggttacccaagtggaattggtaatcttcagcagttgagagttcttgatttgcataataatgtgttatggggtgatgttggggagttgttcttggagttgaagtatatagagcatcttgatttgagtaacaactcgttttttgggtcgcttcctatgaacccggaaaatggatcatttggttcaacaattcatgtaatgaatttgagtcataacaatttggatggtggtttttttcctggaaaattgttgcagacatttgggaatttgcaggttttggatttggggtataatggtttaacgggggagcttccttcgttcatgtttttgtataatttaagggttctaaggcttgggaataatcagctttatgggttgattcctgaggagcttttgcagggaatgggtccattggaagaactggatcttagtggcaatggattttcaggttcaattcctgaagtgaattcaacaaagttgagggttctgaatatttcgtcgaaccacctattaggttcattgccatcttctgtaggaaattgtgcagttgtggatttgagcaaaaatgtgctacatgatgatatatcagttattgagagctgggaaaccaatttggaaattattgatttaagttcaaataggttaactggaaatattcccaatatcatcgctcagtttcaacaattgacttcaattaatttcggaaacaattctcttgagggaatgttgccttcagcactgggcacttcaccaagactggttaaacttgatctcagcaccaataaacttggtggacctattccaactacgtattttacttcgacaacactgatgaacctaaatgtatctggaaatcggttgtcaggatcaattcctcttgaaggatctcatgctagcgaattgcttgttcaatcaccataccaggttgctctagagtctcttgatctttcagaaaacacattgacaggtaatttgtcttctgccattggtaatctgaggaggcttcatgtgctaaatcttgctaagaatcagttatcaggcatgctgcccactgaattggctgaacttagaaacttggaattcctggatgtatctaataacaattttagtggtaggatccctgaaaagctttcattgaatttgagggtgtttgatgtgtccaacaatgacttaagtggtgcaatccctgataatttgaaaaactttaatgaaagttcatttcgtcctggaaactcaggtttggtaatcccagaagactggtcacatcataatcctggtgatcatgatcaaaacagtcagcaccatcataactcgaaatccagtatcagggtagcaatcattcttgcctcagttggagctgctttaatgattggtgttgttctccttgcttaccatcgagaacggttccaagatatccgcttgccaagtggatttaaaggccagtctggtggaagcgatgttaaacttgggaggttcagccggcctgctattttcaagttccatggctcctcagaaccgccgccaacttcgttgagtttctctaatgatcacttgctcactgcgaactcgagatctctgtctgggcaaattgagtcaggtacagaaattgttgagcatgtttctccagagggagtaactgcagtttcagcatctacacatcttggtgctgtgggtaatcatcctgcaccatctggtcggagatcctctccaggctctccaatggcttcctcacctcgttttatcgacacaattgaacaaccggtgacaatggatgtgtactctccggatagattggctggggaattgttcttcctggatggttcactgccatttactgccgaggagttatctcgtgcccctgctgaagttctcggtagaagtagccatggtacattgtataaagctactctgaataatggatgcgttcttactgtcaagtggttgcgggttgggttagtaaaaaacaagaaagaatttgcaaaggaagttaaaaagattggatctattaggaaccccaatgctgttcctttacgagcatattactggggacctcgggaacaagagaggcttattttagcggactacataccaggagacagtttggcgttgcatctctatgagacaaccccacgaaggtactctccgttgtccttcaaccaaaggttaaaagtagctgttgaagttgctcggggtttgggatatctgcatgagagaggactgcctcatggaaacttgaagcctactaatataatcttggttggtgctgattacagtgccctcctcactgattatggtctccatcgcttgatgacttcagcagggattgcggagcagatactgaacctaggggctctaggttaccgtgctcctgaacttgccaccgcgaccaaacctatcccatcatttaaggctgatgtatatgcccttggtgtgattctaatggaattgctgacgagaagaagtgcgggtgacataatctcggggcaatctggtgctgttgatttaacagactgggttcggttgtgtgatcaagaaggtagaggaatggactgtattgatagggacattgcaggaggggaagaacactgcagagcaatggacgatctgctaactgtgtcgttaaggtgtattcttccggtaaatgaaaggcctaacattagacaagttgttgaagatctgtgttccatatctgtgtga

SEQ ID NO: 32 - protein, 1059 aa, Capsicum annuum
MTPPQSLSLLLLALVFMLNGCSSAVEDEIRSLLEFKKGIKNDPLGKIFSSWIPSPSDLSACPSSFYGVECDANSNSVASITLDGLGLVGDLKFSTLNGLKQLKVLSLSGNSFTGRIVPALGTLVTLQHLDLSGNLFYGPIPARINELWGLNYLNLSNNNFNGGYPSGIGNLQQLRVLDLHNNVLWGDVGELFLELKYIEHLDLSNNSFFGSLPMNPENGSFGSTIHVMNLSHNNLDGGFFPGKLLQTFGNLQVLDLGYNGLTGELPSFMFLYNLRVLRLGNNQLYGLIPEELLQGMGPLEELDLSGNGFSGSIPEVNSTKLRVLNISSNHLLGSLPSSVGNCAVVDLSKNVLHDDISVIESWETNLEIIDLSSNRLTGNIPNIIAQFQQLTSINFGNNSLEGMLPSALGTSPRLVKLDLSTNKLGGPIPTTYFTSTTLMNLNVSGNRLSGSIPLEGSHASELLVQSPYQVALESLDLSENTLTGNLSSAIGNLRRLHVLNLAKNQLSGMLPTELAELRNLEFLDVSNNNFSGRIPEKLSLNLRVFDVSNNDLSGAIPDNLKNFNESSFRPGNSGLVIPEDWSHHNPGDHDQNSQHHHNSKSSIRVAIILASVGAALMIGVVLLAYHRERFQDIRLPSGFKGQSGGSDVKLGRFSRPAIFKFHGSSEPPPTSLSFSNDHLLTANSRSLSGQIESGTEIVEHVSPEGVTAVSASTHLGAVGNHPAPSGRRSSPGSPMASSPRFIDTIEQPVTMDVYSPDRLAGELFFLDGSLPFTAEELSRAPAEVLGRSSHGTLYKATLNNGCVLTVKWLRVGLVKNKKEFAKEVKKIGSIRNPNAVPLRAYYWGPREQERLILADYIPGDSLALHLYETTPRRYSPLSFNQRLKVAVEVARGLGYLHERGLPHGNLKPTNIILVGADYSALLTDYGLHRLMTSAGIAEQILNLGALGYRAPELATATKPIPSFKADVYALGVILMELLTRRSAGDIISGQSGAVDLTDWVRLCDQEGRGMDCIDRDIAGGEEHCRAMDDLLTVSLRCILPVNERPNIRQVVEDLCSISV

SEQ ID NO: 33 - DNA, 2574 bp, Cucumis sativus
atgggaaagggtcactaccctctgagtgaatttcaggtatacccatttcagttcatgcagattcttccatcaaaaactgctgcttggtgctctaaatcaccaaataacaacaggaaactcaagaaccaacaatggagaagctgttcttcgtacctcattgctctggcttttgctcttcttttggcttgttcctttcggccagtcactggccagatgtgggatggagtgtctgttactcaaggcgattttcaagctcttcaagctataaaacatgaactcgttgatctaaaaggagttttgcgtagttggaatggaagtaatggagcttgttccgggcagtgggtaggaatcaagtgcgtgaaagggcaggttattgctatacagcttccatggaaggcccttgccggcaggatttctgataggattgggcagctccgggaacttcggaagcttagcctccatgacaatgtgatctctggcgtgatccctcggtccatcgggttccttccaaatctcagaggaatttatcttttcaacaacagattatcaggttctattcctcctactattggtcatcttcccctacttcaaactcttgatttgagcaacaatttgctcactggagaaattcctttcggtatagcaaattctaccaaattgattagagttaatctgagttacaattcattgtcgggatctatcccaacaagtttcacgcagtcgttttctcttataattctcgcccttcagcacaataatatttctgggactgtcccagattcttggggaagtcttggaaacaaaacttgcccacttggggttttgacacttgatcataatgctatatctggagcaattccagcttctttaactaaattggaatggcttcaagagatttccatcagtgagaacaagatctctggagctataccaggtgaaataggcaggctgaagagacttcgtcttttggatttgtcgaataacgccattaacggcagcttcccttcaagtttctccaatctctcctctcttcagttattgaaggttgaaaataatcgtcttgaaagccagattccagaagatattgatagattgcacaatctctcggtggtcaagcttgggaagaatcgttttagtggtgagattcctgcaagttttgggaacatttctgctattagccaacttgatttctcagaaaataacttcactggacagattccaacatcgttgacccggttgttgaatctaacctctttcaatgtctcttacaacaatctctctggccctgtcccagttctcctatcaaataagttcaatgcgagctcttttgtagggaaccttcaactgtgtggttttagtacttcaactccatgcctacctgcatcctccccacagaatattactactccatcaactgaggtcctgaaaccgcgtcaccaccgcagacttagtgtaaaggacataatcctgatagcggcaggagctctgttgctgcttcttcttcttctctgctcaattttgctctgttgtttattatcaaaaagagcagctgccaggaagactgacaagacaactgccaagcaagctgctgccagaagcatcgaaaaggcagcacccgggagcacagaagttggagcaggtgaagctggtggaaaactagttcattttgatgggccatttgttttcactgctgatgatcttctgtgtgcgactgctgagatcatgggcaagagcacatatggaacagcatataaagcaacattggaagatggaaatgaagttgcagtgaaaagattgagagagaaaacaacaaaagggcacaaggaatttgaaaccgaagttgcaggcctcggaaaaattcgacatccaaatctacttgctcttagagcttactacttaggccccaaaggagagaagctcctggtctttgattacatgcctagaggaagcctctcttccttccttcatgctcgtggaccagaaaccacagtcgattggccaacaaggatgaaaatagcgattggaatcacgcaaggcttgaactacctccacacagaggagaatcttattcatggaaacctcacgtccagcaacattctgctggatgatcagagtaatgctcgtattgcagattttgggcttcccaagctcatgacttcagctgcagccacaaatgtgattgccactgcaggatcacagggctacaatgcaccagagctaacaaaaaccaaaaagaccacaacaaagacagatgtctacagccttggagtcataatattggaactcttgacaggaaaatctcctggagaagccatggatggaatggacttaccacagtgggtggcctcaatagtaaaggaagagtggacaaatgaagtgtttgatttggagcttatgaaagatacacaaaatattggcgatgaactgctgaacactttaaaactagccctgcattgtgttgatccgtcaccaactgctcggccagatgttcagcagattctccagcaactggaggaaatcaatgcttctacttccggtgacgacggtgccaaaaatcaaccagaaaatgaatag

SEQ ID NO: 34 - protein, 857 aa, Cucumis sativus
MGKGHYPLSEFQVYPFQFMQILPSKTAAWCSKSPNNNRKLKNQQWRSCSSYLIALAFALLLACSFRPVTGQMWDGVSVTQGDFQALQAIKHELVDLKGVLRSWNGSNGACSGQWVGIKCVKGQVIAIQLPWKALAGRISDRIGQLRELRKLSLHDNVISGVIPRSIGFLPNLRGIYLFNNRLSGSIPPTIGHLPLLQTLDLSNNLLTGEIPFGIANSTKLIRVNLSYNSLSGSIPTSFTQSFSLIILALQHNNISGTVPDSWGSLGNKTCPLGVLTLDHNAISGAIPASLTKLEWLQEISISENKISGAIPGEIGRLKRLRLLDLSNNAINGSFPSSFSNLSSLQLLKVENNRLESQIPEDIDRLHNLSVVKLGKNRFSGEIPASFGNISAISQLDFSENNFTGQIPTSLTRLLNLTSFNVSYNNLSGPVPVLLSNKFNASSFVGNLQLCGFSTSTPCLPASSPQNITTPSTEVLKPRHHRRLSVKDIILIAAGALLLLLLLLCSILLCCLLSKRAAARKTDKTTAKQAAARSIEKAAPGSTEVGAGEAGGKLVHFDGPFVFTADDLLCATAEIMGKSTYGTAYKATLEDGNEVAVKRLREKTTKGHKEFETEVAGLGKIRHPNLLALRAYYLGPKGEKLLVFDYMPRGSLSSFLHARGPETTVDWPTRMKIAIGITQGLNYLHTEENLIHGNLTSSNILLDDQSNARIADFGLPKLMTSAAATNVIATAGSQGYNAPELTKTKKTTTKTDVYSLGVIILELLTGKSPGEAMDGMDLPQWVASIVKEEWTNEVFDLELMKDTQNIGDELLNTLKLALHCVDPSPTARPDVQQILQQLEEINASTSGDDGAKNQPENE

SEQ ID NO: 35 - DNA, 2490 bp, Cucumis sativus
atgggtaaaactcaaactccatttgatttcaactctttttcgagatacccatttggggattttgggaagaaggagaaatggaagaatctgagacaaaaccctaacgtgtttgttttgttgcttttgttgcttaatttggttcctgttctgagtcaggattgggatggtgtggttgtgactcaagctgatttccaatcccttcaggcatttaagcaagagcttgatgatcccaaagggtttttgaagagttggaatgatagtggatttggggcttgttctggtggttgggctgggattaaatgtgccaagggacaagttattgtaatacagcttccatggaagggattaggaggtagaatcactgagaagattggccaacttcaagcacttaggaagcttagtttacatgataactctattggtggatcaatcccttcttcattggggcttcttccaaatcttagaggggttcagttattcaataatcgactttcgggttcgattccggcttcattgggactttgtcccgtacttcaaacacttcacattagtaacaatttgctcactggaacaataccacctactttggctaactccaccaagctttattggcttaacttgagcttgaactcgctttcgggtccgattccaactactcttactcgttcggtgtcccttactttccttgaccttcaacataacaatctctcaggcagcattccagactcttggggtggtgacgagcaaaaccgtgtgtttcagctcaagtctctgactcttgatggcaatctcctctcaggtaccataccaacttctttaagtaagttaagtgagcttcaagtgatttcccttagccacaataggttaaatggtggcattcctgaagaaattagtaggctttctctgctcaaaacattggatgtttctaataatttccttaatggaagtatgccccaaagttttgatagattgagaaacctctctatactcaacttgagtagaaacagattcaatggtcaaattcctgaaactttagggaacgtttcaacacttaaacaacttgatttatctcagaacaatctaagtggagaaattccagcttcccttgctgatttgcaaggtcttcaatccttaaatgtctcatacaacaacctttcgggttcggttcctcgagctctcgctgaaaaattcaatgcaagttcctttgttgggaaccttcagctctgtgggttcagtggttcaattctgtgcccgtccccagccccttctcaagaagctcctgctcctccaccagaaagttcgacaacccgacatcggaagttaagtactaaagatataatcctcatagcagcaggagccctccttctggtattggttatagttttctttatattgctctgctgcttgataaggaaaagggctgcttcaaaagggaaggatggtggtgaagctggagcagctggggctgcgagggccgagaagggcgtccccccgactagcagtgaagttgaagcggctggaggtggggatgctggtgggaagctcgtccatttcgacgggcaaacggtcttcacagcagatgatcttttgtgtgcaacagcagaaataatgggaaagagtacttatgggactgtttacaaggccacgttggaggatggaaatcaagttgcggtgaagagattgagagagaaaatcactaaaagccaaaaggaatttgaagcagaggtcaacatacttggcaagatcagacatccaaatctattagctttaagagcttattacttgggacccaaaggagagaagcttctcgttttcgattatatgccgaatggaagtctcgcgacgttcctgcatgctcgtggaccggacacgtccatcgattggccaacaaggatgaagatagctcagggtatgacacgtggcctgtgtcaccttcacacgcacgagaacagcatccatggaaacctgacatcaagcaacattctactagatgagtacataaacgctaagattgcagattttggcttgtcaaggttgatgacagcagctgcaagctcaaatgtaatagcaacagccggggcacttgggtaccgagcaccagaactctcaaagttgaagaaagcaaacacaaaaaccgatatttacagccttggagtcatcatattggaactcttgactggtaagtcccccggtgaagccatgaacggagttgatttacctcaatgggtcgcatcgatagtcaaagaggagtggactaatgaagtttttgatttggaactgatgagagatgcatctaccattggagatgagttactaaacactctgaaactcgctttgcattgtgtcgacccttcaccgtcagcgagaccggaagttcaacaagttttgcaacaactggaagagataaggccagaaactgctccaagttctggggatgatggaataggaataggaggaggaggaggaggagccccttcaacaagtgagtga

SEQ ID NO: 36 - protein, 829 aa, Cucumis sativus
MGKTQTPFDFNSFSRYPFGDFGKKEKWKNLRQNPNVFVLLLLLLNLVPVLSQDWDGVVVTQADFQSLQAFKQELDDPKGFLKSWNDSGFGACSGGWAGIKCAKGQVIVIQLPWKGLGGRITEKIGQLQALRKLSLHDNSIGGSIPSSLGLLPNLRGVQLFNNRLSGSIPASLGLCPVLQTLHISNNLLTGTIPPTLANSTKLYWLNLSLNSLSGPIPTTLTRSVSLTFLDLQHNNLSGSIPDSWGGDEQNRVFQLKSLTLDGNLLSGTIPTSLSKLSELQVISLSHNRLNGGIPEEISRLSLLKTLDVSNNFLNGSMPQSFDRLRNLSILNLSRNRFNGQIPETLGNVSTLKQLDLSQNNLSGEIPASLADLQGLQSLNVSYNNLSGSVPRALAEKFNASSFVGNLQLCGFSGSILCPSPAPSQEAPAPPPESSTTRHRKLSTKDIILIAAGALLLVLVIVFFILLCCLIRKRAASKGKDGGEAGAAGAARAEKGVPPTSSEVEAAGGGDAGGKLVHFDGQTVFTADDLLCATAEIMGKSTYGTVYKATLEDGNQVAVKRLREKITKSQKEFEAEVNILGKIRHPNLLALRAYYLGPKGEKLLVFDYMPNGSLATFLHARGPDTSIDWPTRMKIAQGMTRGLCHLHTHENSIHGNLTSSNILLDEYINAKIADFGLSRLMTAAASSNVIATAGALGYRAPELSKLKKANTKTDIYSLGVIILELLTGKSPGEAMNGVDLPQWVASIVKEEWTNEVFDLELMRDASTIGDELLNTLKLALHCVDPSPSARPEVQQVLQQLEEIRPETAPSSGDDGIGIGGGGGGAPSTSE

SEQ ID NO: 37 - DNA, 3186 bp, Cucumis sativus
atgagggccttgaggattttggcggtgtccttcatgcttgtttctgctatggctcagctcccttcacaagatatattggctttgcttgagttcaagaagggtatcaagcatgacccaacagggtttgttgttagctcatggaatgaggagtccattgattttgatggctgtccttcttcttggaatgggattgtttgcaatagtggaagtgttgctggagtggtgttggatggtttgggcctttctgctgatgtagatttgaatgtgttttcaaacctaacaaaacttgcaaagctttccttgtcaaacaactcgataacagggaagatgccggacaatatagccgagtttcaaagtctagagtttcttgatatctcaaataacctcttttcttcatccttaccacaagggtttggtaggttaaccagcttgcagaacctatcattggctggcaacaacttctcgggtaacatcgatccaattgctgatcttcaatcaattcgatcattagatttaagtcacaactcattttctggctctctgcctacagcattgactaagctgacaaacttggtatacctggatctatctttcaatggttttactgacaggattccaaagggttttgagctcctttcagagcttgaggtgcttgacttgcatggtaatatgcttgatggcacactagatgttgaatttttcactttgtctggtgctactcatgttgacttcagtaacaacatgctaacgagctctgacatggggcatgggaagttcttgcctcgactttctgattccatcaagcatttaaacctcagtcacaaccagcttactggatcgttggttaatggaggcgagttatcactgtttgaaaacttgaaaacattggatttaagttacaatcagttttcaggagagctacctggttttagttttgtgtatgatctccaaattttgaaactgagcaacaatagattctctggagatattcctaataacctattaaaaggagatgcttcagttttaactgaactggatttgagtgccaacaatctctcagggccagtaagtatgatcacatcaaccaccttacttgtcctcaatctttcgtccaatcagcttaccggtgaacttccattgctgaccggaagttgtgctgttcttgatctctctaataaccaatttaagggaaatttgacaaggatgataaagtgggggaaccttgaattccttgatcttagtcagaatcttttgacagggccaatccctgagttaactccacagttcttgcggttaaactttctgaacttatcccataatactctgagtagttcactcccaagtgctattaccaagtatccaaagcttcgagtccttgatcttagctctaaccagtttgatggccctttgctggctgacttgctcacaatgtccactttggaagagctctatctggaaaataatttgctcaacggtgctgttaagttcttgcttccttcccctggtaaagcaaacctcgaggttcttgatctttctcataatcagctcgatggctattttcctgacgaatttgtatctttaactggccttacaatgctgaatattgctggaaacaatttttctggatccttgcctacatccatgtccgatctgagtgcattgatctcattagacatgtcacaaaatcatttcacaggtccccttccaagcaacttgtctagtgacattcaaaactttaatgtttcatctaatgatctttcgggaactgtccccgaaaatttgagaaaatttccacgttctgcattctttcctggaaattcaaaactgaatcttccaaatggtcctggatcaagcaataatcaagatggtagatcagggagaaagaaaatgaacacaattgtaaaagtaataataattgtttcatgtgtaattgcattggttattattgtcctccttgctatcttcttccattacatctgcatatcaaggaaaaatccacctgagcttgccagtactaaagacactcgtagacatagttctctaagctcctcggccattggcggaactggagctggtagcaatctggttgtttcagctgaggaccttgtgacttcaaggaaagggtcgtcgtctgagataatcagccccgatgagaaactagcagtaggaactggtttctccccagcaaaaaacagccatttctcttggtcgcctgaatcaggtgattcatttactgctgaaaaccttgcaagattggatgtgagatcaccggatcgtttggttggtgaacttcattttcttgatgattccatctcattgacaccagaggagctatcaagagctccagctgaagtgttagggagaagcagccatggcacttcttacagagcaacactagagagtgggatgtttctgacagttaagtggttgagggaaggtgtagcgaagcagagaaaggaatttgctaaggaggctaaaaagtttgcaaatatcaggcatccgaatgtcgttggcttgagaggatattattgggggcctactcagcatgagaagctcattctttcggactatatctcacctggaagccttgcagtctttctatatgatcgtccgagtagaaaaggtcctttgacatgggctcaaaggctcaagatagctgttgatatagcacgtggcctgaactatctccatttcgatcgagctgttccacacggaaatcttaaagcaacaaatgtactactagatggagcagacctgaatgctcgagttgctgattattgccttcatcgcctcatgactcatgctggcactattgaacaaattctcgacgctggggtcttaggctaccgtgcaccggaattggcagcttccaaaaagccacagccctcattcaagtctgatgtctatgcatttggagtgatacttctggaacttctaactggaagatgtgctggtgatgtgatctctggtgaagagggaggagttgatctaacagactgggtgcggttacgggtggccgaagggcggggctccgactgtttcgataccttattgttacccgaaatgagcaatgcggcagcagagaagggtatgaaggaggtgcttggtatagctttacgatgtattcgaacagtatcagaaagaccaggtatcaagactatatacgaggatctttcctccatatag

SEQ ID NO: 38 - protein, 1061 aa, Cucumis sativus
MRALRILAVSFMLVSAMAQLPSQDILALLEFKKGIKHDPTGFVVSSWNEESIDFDGCPSSWNGIVCNSGSVAGVVLDGLGLSADVDLNVFSNLTKLAKLSLSNNSITGKMPDNIAEFQSLEFLDISNNLFSSSLPQGFGRLTSLQNLSLAGNNFSGNIDPIADLQSIRSLDLSHNSFSGSLPTALTKLTNLVYLDLSFNGFTDRIPKGFELLSELEVLDLHGNMLDGTLDVEFFTLSGATHVDFSNNMLTSSDMGHGKFLPRLSDSIKHLNLSHNQLTGSLVNGGELSLFENLKTLDLSYNQFSGELPGFSFVYDLQILKLSNNRFSGDIPNNLLKGDASVLTELDLSANNLSGPVSMITSTTLLVLNLSSNQLTGELPLLTGSCAVLDLSNNQFKGNLTRMIKWGNLEFLDLSQNLLTGPIPELTPQFLRLNFLNLSHNTLSSSLPSAITKYPKLRVLDLSSNQFDGPLLADLLTMSTLEELYLENNLLNGAVKFLLPSPGKANLEVLDLSHNQLDGYFPDEFVSLTGLTMLNIAGNNFSGSLPTSMSDLSALISLDMSQNHFTGPLPSNLSSDIQNFNVSSNDLSGTVPENLRKFPRSAFFPGNSKLNLPNGPGSSNNQDGRSGRKKMNTIVKVIIIVSCVIALVIIVLLAIFFHYICISRKNPPELASTKDTRRHSSLSSSAIGGTGAGSNLVVSAEDLVTSRKGSSSEIISPDEKLAVGTGFSPAKNSHFSWSPESGDSFTAENLARLDVRSPDRLVGELHFLDDSISLTPEELSRAPAEVLGRSSHGTSYRATLESGMFLTVKWLREGVAKQRKEFAKEAKKFANIRHPNVVGLRGYYWGPTQHEKLILSDYISPGSLAVFLYDRPSRKGPLTWAQRLKIAVDIARGLNYLHFDRAVPHGNLKATNVLLDGADLNARVADYCLHRLMTHAGTIEQILDAGVLGYRAPELAASKKPQPSFKSDVYAFGVILLELLTGRCAGDVISGEEGGVDLTDWVRLRVAEGRGSDCFDTLLLPEMSNAAAEKGMKEVLGIALRCIRTVSERPGIKTIYEDLSSI

SEQ ID NO: 39 - DNA, 3108 bp, Cucumis sativus
atggcttcggagccggagactctctctcttaacttcatttatctgcttatcgtcttggtttcttcagcttctgattctgaactaaactgtctgcttgaattcaagaaggggattcagaaagatccgcacaactcggtcaacgggaagtgggatttggcttccgtttcgaactttgatggttgtccttcgtcctggactggtgtgtcttgtgatgagaacggtaatgtgtctgcaattgtgctggaccggcttggcttgggaggggagttgaagtttcagactctgattgggcttaagagtcttaagaatttgagtctttttggaaatgattttactggacggcttgttccggctcttgggacattatctaatctgcagcatttggatctgtcctcgaatagattttatgggccgatcccggagcggatcaatgatctttacaatctgaactatcttaatttctcagcaaacgagttcaatggtgggtttccagttggtagattgaatcttaatcagctcaaggtgttggatttgcactctaatcgactttatgggaacattggcctgctggtttcccagctgcggaatgtggaatatgttgatttaagccacaatgaattctacggcggactttcaattggccctgataacgtctcaagtctggctaatacattgaaaagtttcaacttaagttacaatagattgaatggtggattctttgacgttgactctctcatgttatttcgaaatttggtagtcttggatatgggtcataaccagattataggggaattgccttcatttgggtccttgcctaatttgcgcgttttgaggcttggttataatcttttatctggctcggtgcctggggaactgttaaacaggtccttgcaattggaggaattggatcttagtggtaatgcatttacaggttcaaatcttcgggttgactcctctactttgaaatttttggacctctcttcaaataatttgtcgggagacatatcagttttgcaatcttgggaagccaattttgaagttcttgatttaagttcaaataagttctcgggaagcttcccaaacataacttccttctttcagggattgaaggtgcttaatgtcagaaataattccttagaaggccctttgccgtttacattggtgaactatcctagcatgtctgcagttgacttcagtttaaatggttttagcggtactgtccctgctagtttctttacatctgttaccttgatcagcctcaatctgtccggaaaccggctaaccggtcccattccccttcaaggctcaagtgttagcgagttgttagttaaaccatcagatctgccattggaatatcttgatctatccaataactcgttgattggtgggttgccttctgaaatagataaactggcgaggctcaaattgctaaatcttgctaagaatgaattatcagggccacttccagatcaattgaccagattgagtaacttggagtaccttgatttatcaaacaacaaatttacaggtgaaattcctggtatgcttcccgacctacatgtttttaatgtatcctacaatgatctctcaggtgacgttccagataatttaaggaacttccccatctcatcatttcgtccaggaaatgataagcttaacttaccaaaagagataggttcagagaactcgattccaaataatttccctgagcatggaagacgtcgtacttctaaagctaatatccaaatagctattattcttgcctcagttggagcagttgtgatgattgtttttcttctacttgcttatcatagagcacaacttaaagagttccatggaagaagtatatttagcggccaaggtactgaaaggaacattaagatagaacgtttcaggccttccattttcaagttccaaccaaacaatcagcctccaccaacctcttcaagtttttcaaatgaccatttactaacctccacttcaaggacattatctgggcaggcagaattctcttctgagatttctgaacatgttttacctggaggtgctgcagcgagttcatcaatgattattcctaatttactcgacgatcctgttacttctgggaaaaattcctccccaggttccccattatcttcctcacaccagtttgttgacgggcgtgaacaacctgtgacactagatgtgtattcaccagatcgtttagctggagaattgttttttctggacaattcactgctattcactgctgaggaactatccagagccccagctgaagttctcggtagaagcagccatggaacactatataaagctactctggatagtggacatatgctggctgttaagtggttacgtgtggggctggtcaaacataagaaggaatttgccaaggaagttaaaagaataggatcaatgaggcataaaagcattgttcctttacgagcatattattggggtccaagagaacaagaaagacttcttttagctgactatattttgggagatagcttagctctacatctttacgaaactacaccacgaaggtattctcggttatcgttcagtcaacgacttaaaattgcagtggaagttgctcgctgtctgttataccttcatgataggggcctccctcatggaaacttaaagccaacaaatattatcttggcaggccatgattccgatgcccggctcactgactacggacttcaccgcttgatgacaccagcaggcattgcggagcagatattgaatctaggggcacttggatattgcgctccagaactcgcttgtgcagcaaaacctggtccatctttcaaggctgacatttattcatttggggtaattttaatggagcttttaaccaaaagaagtgcaggcgacataatatcgggtcaatcgggggccgtcgatctcacagattgggtgcgtttatgcgatcaagaaggacgaagaatggactgtatagaccgagatatcgtagttggagaagagccttcgaaagctatggatgaactgttgggtgtatctctcaagtgcattcgtcctgtaaatgagaggcctaacatcagacaagtgtttgatgatttatgtgctatatgtgtttga

SEQ ID NO: 40 - protein, 1035 aa, Cucumis sativus
MASEPETLSLNFIYLLIVLVSSASDSELNCLLEFKKGIQKDPHNSVNGKWDLASVSNFDGCPSSWTGVSCDENGNVSAIVLDRLGLGGELKFQTLIGLKSLKNLSLFGNDFTGRLVPALGTLSNLQHLDLSSNRFYGPIPERINDLYNLNYLNFSANEFNGGFPVGRLNLNQLKVLDLHSNRLYGNIGLLVSQLRNVEYVDLSHNEFYGGLSIGPDNVSSLANTLKSFNLSYNRLNGGFFDVDSLMLFRNLVVLDMGHNQIIGELPSFGSLPNLRVLRLGYNLLSGSVPGELLNRSLQLEELDLSGNAFTGSNLRVDSSTLKFLDLSSNNLSGDISVLQSWEANFEVLDLSSNKFSGSFPNITSFFQGLKVLNVRNNSLEGPLPFTLVNYPSMSAVDFSLNGFSGTVPASFFTSVTLISLNLSGNRLTGPIPLQGSSVSELLVKPSDLPLEYLDLSNNSLIGGLPSEIDKLARLKLLNLAKNELSGPLPDQLTRLSNLEYLDLSNNKFTGEIPGMLPDLHVFNVSYNDLSGDVPDNLRNFPISSFRPGNDKLNLPKEIGSENSIPNNFPEHGRRRTSKANIQIAIILASVGAVVMIVFLLLAYHRAQLKEFHGRSIFSGQGTERNIKIERFRPSIFKFQPNNQPPPTSSSFSNDHLLTSTSRTLSGQAEFSSEISEHVLPGGAAASSSMIIPNLLDDPVTSGKNSSPGSPLSSSHQFVDGREQPVTLDVYSPDRLAGELFFLDNSLLFTAEELSRAPAEVLGRSSHGTLYKATLDSGHMLAVKWLRVGLVKHKKEFAKEVKRIGSMRHKSIVPLRAYYWGPREQERLLLADYILGDSLALHLYETTPRRYSRLSFSQRLKIAVEVARCLLYLHDRGLPHGNLKPTNIILAGHDSDARLTDYGLHRLMTPAGIAEQILNLGALGYCAPELACAAKPGPSFKADIYSFGVILMELLTKRSAGDIISGQSGAVDLTDWVRLCDQEGRRMDCIDRDIVVGEEPSKAMDELLGVSLKCIRPVNERPNIRQVFDDLCAICV

(19) United States
(12) Patent Application Publication
(10) Pub. No.: US 2020/0239898 A1
(43) Pub. Date: Jul. 30, 2020

(54) METHODS OF INCREASING DISEASE RESISTANCE IN A PLANT

(71) Applicant: THE GOVERNING COUNCIL OF THE UNIVERSITY OF TORONTO, Toronto (CA)

(72) Inventors: David S. GUTTMAN, Toronto (CA); Darrell DESVEAUX, Toronto (CA); Glenn Adam MOTT, Toronto (CA)

(21) Appl. No.: 16/639,576

(22) PCT Filed: Aug. 29, 2018
(86) PCT No.: PCT/CA2018/051038

§ 371 Date: Feb. 16, 2020

Related U.S. Application Data
(60) Provisional application No. 62/552,092, filed on Aug. 30, 2017.
(60) Provisional application No. 62/553,384, filed on Sep. 1, 2017.

(51) Int. Cl. C12N 15/82

(52) U.S. Cl. CPC C12N 15/8218; C12N 15/8282; C12N 15/8283; C12N 15/8281

(57) ABSTRACT

Reducing or eliminating activity of certain leucine-rich repeat, receptor-like kinases (LRR-RLK) polypeptides in a plant results in an increase in immune response in the plant, thereby conferring increased disease resistance in the plant. The genes that encode the polypeptides are in the same phylogenetic clade, and are termed Broad-Range Resistance (BRR) genes. The related LRR-RLK receptors are called Broad-Range Resistance (BRR) receptors.

### CROSS-REFERENCE TO RELATED APPLICATIONS

This application claims the benefit of United States Provisional Patent Applications U.S. Ser. No. 62/552,092 filed Aug. 30, 2017 and U.S. Ser. No. 62/553,384 filed Sep. 1, 2017, the entire contents of both of which are herein incorporated by reference.

### FIELD

This application relates to plant biotechnology, in particular to methods of increasing disease resistance in a plant.

### BACKGROUND

Plants can perceive microbes through pattern recognition receptors that recognize conserved microbe-associated molecular patterns (MAMPs). The recognition of MAMPs by immune receptors elicits an innate immune response that protects the host from infectious diseases. The genome of plants comprises a class of genes that encode leucine-rich repeat, receptor-like kinases (LRR-RLKs), which are generally involved in pathogen detection or developmental processes. Normally the loss of an immune-related LRR-RLK dampens the immune response, and is therefore detrimental to plant disease resistance. Further, loss of an immune-related LRR-RLK is often detrimental to proper development in the plant. Furthermore, an increase in plant immune response is generally coupled to a loss in productivity or yield.

There remains a need for methods of improving immune response in a plant to provide a broad range of pathogen resistance, while not unduly affecting plant development processes.

### SUMMARY

In one aspect, there is provided a method of obtaining a plant with increased disease resistance, the method comprising: determining whether a species of plant has a wild type gene that encodes a functioning leucine-rich repeat, receptor-like kinase (LRR-RLK) polypeptide having at least 50% percent sequence identity to the amino acid sequence of a homologous LRR-RLK polypeptide in Arabidopsis thaliana; identifying a plant having reduced or eliminated functioning of the LRR-RLK polypeptide by (a) in a population of the species, identifying a plant that contains a mutated gene instead of the wild type gene and determining whether the mutated gene in the plant encodes a different polypeptide than the functioning LRR-RLK polypeptide, or (b) silencing expression of the wild type gene or the functioning LRR-RLK polypeptide; and, confirming that the plant with the reduced or eliminated functioning of the LRR-RLK polypeptide has an increased immune response or a decreased disease severity upon pathogen challenge compared to a wild type plant, thereby obtaining a plant with increased disease resistance.

In another aspect, there is provided a method of obtaining a plant with increased disease resistance, the method comprising: determining whether a species of plant has a wild type gene that encodes a functioning leucine-rich repeat, receptor-like kinase (LRR-RLK) polypeptide having at least 50% percent sequence identity to the amino acid sequence of a homologous LRR-RLK polypeptide in Arabidopsis thaliana; identifying a plant in a population of the species of plant containing a mutated gene instead of the wild type gene; if the plant contains the mutated gene instead of the wild type gene, determining whether the mutated gene in the plant encodes a different polypeptide than the functioning LRR-RLK polypeptide; and, confirming that the plant with the mutated gene has an increased immune response or a decreased disease severity upon pathogen challenge compared to a wild type plant, thereby obtaining a plant with increased disease resistance.

In another aspect, there is provided a method of obtaining a plant with increased disease resistance, the method comprising: determining whether a species of plant has a wild type gene that encodes a functioning leucine-rich repeat, receptor-like kinase (LRR-RLK) polypeptide having at least 50% percent sequence identity to the amino acid sequence of a homologous LRR-RLK polypeptide in Arabidopsis thaliana; silencing expression of the wild type gene or the functioning LRR-RLK polypeptide in a plant of the species; and, confirming that the plant with the silenced expression has an increased immune response or a decreased disease severity upon pathogen challenge compared to a wild type plant, thereby obtaining a plant with increased disease resistance.

In another aspect, there is provided a method of increasing disease resistance in a plant, the method comprising: attenuating or eliminating activity in the plant of a functioning leucine-rich repeat, receptor-like kinase (LRR-RLK) polypeptide, or silencing expression in the plant of a functioning polynucleotide encoding the functioning LRR-RLK polypeptide, the functioning LRR-RLK polypeptide having at least 50% percent sequence identity to the amino acid sequence of a homologous LRR-RLK polypeptide in Arabidopsis thaliana; and, confirming that the plant having the attenuated or eliminated activity or the silenced expression has an increased immune response or a decreased disease severity upon pathogen challenge compared to a wild type plant of the same species, thereby producing a plant with increased disease resistance.

In another aspect, there is provided a method of increasing disease resistance in a plant, the method comprising: silencing expression in a plant of a polynucleotide encoding a functioning leucine-rich repeat, receptor-like kinase (LRR-RLK) polypeptide having at least 50% percent sequence identity to the amino acid sequence of a homologous LRR-RLK polypeptide in Arabidopsis thaliana; and, confirming that the plant having the silenced expression has an increased immune response or a decreased disease severity upon pathogen challenge compared to a wild type plant of the same species, thereby producing a plant with increased disease resistance.

In another aspect, there is provided a method of increasing disease resistance in a plant, the method comprising: attenuating or eliminating activity in the plant of a functioning leucine-rich repeat, receptor-like kinase (LRR-RLK) polypeptide having at least 50% percent sequence identity to the amino acid sequence of a homologous LRR-RLK polypeptide in Arabidopsis thaliana; and, confirming that the plant with attenuated or eliminated activity of the LRR-RLK polypeptide has an increased immune response or a decreased disease severity upon pathogen challenge compared to a wild type plant of the same species, thereby producing a plant with increased disease resistance.

In another aspect, there is provided a disease resistant plant comprising a LRR-RLK polynucleotide having silenced expression of a functioning leucine-rich repeat, receptor-like kinase (LRR-RLK) polypeptide, the LRR-RLK polypeptide having at least 50% percent sequence identity to the amino acid sequence of a homologous LRR-RLK polypeptide in Arabidopsis thaliana.

It has now been found that reducing or eliminating activity of certain polypeptides in a plant results in an increase in immune response in the plant, thereby conferring increased disease resistance in the plant. The polypeptides are part of a family of leucine-rich repeat, receptor-like kinases (LRR-RLK). The genes that encode the polypeptides are in the same phylogenetic clade, and are termed Broad-Range Resistance (BRR) genes. The related LRR-RLK proteins are called Broad-Range Resistance (BRR) receptors. The polypeptides have at least 50% percent sequence identity to the homologous LRR-RLK polypeptide in Arabidopsis thaliana (i.e. SEQ ID NO: 6 (AtBRR3), SEQ ID NO: 8 (AtBRR4), SEQ ID NO: 10 (AtBRR5) or SEQ ID NO: 12 (AtBRR6)).

Further features will be described or will become apparent in the course of the following detailed description. It should be understood that each feature described herein may be utilized in any combination with any one or more of the other described features, and that each feature does not necessarily rely on the presence of another feature except where evident to one of skill in the art.

### BRIEF DESCRIPTION OF THE DRAWINGS

For clearer understanding, preferred embodiments will now be described in detail by way of example, with reference to the accompanying drawings, in which:

FIG. 1 depicts a phylogenetic tree of 227 candidate immune receptors identified in the genome of Arabidopsis thaliana. Dots around a perimeter of the tree indicate 169 genes for which T-DNA knock out lines were examined. The highlighted wedge shows a clade of related receptors termed Broad-Range Resistance (BRR) genes.

FIG. 2A depicts a graph of bacterial density (cfu/cm²) in Arabidopsis plants challenged with Pseudomonas syringae DC3000. Bacterial colonization was measured 3 days post inoculation. Knockouts in AtBRR3, AtBRR4, AtBRR5, and AtBRR6 genes (gray bars) had significantly lower bacterial accumulation than wild type Col-0 controls (white bars) and knockouts in AtBRR1 and AtBRR2 genes (black bars).

FIG. 2B depicts a graph of bacterial growth in Arabidopsis plants challenged with Pseudomonas syringae DC3000 extending the bacterial growth analysis to first generation (F1) progeny that are heterozygous in both loci. Bacterial colonization was measured 3 days post inoculation. Data is normalized and shown as fold growth compared to the average bacterial load of the matched wild type control. The legend at the right of the graph read from top to bottom correspond to the bars in the graph read from left to right.

FIG. 2C depicts a graph of sporangiophore count on cotyledons of Arabidopsis plants challenged with Downy Mildew (H. arabidopsidis). Arabidopsis plants with knockouts in AtBRR3, AtBRR4, AtBRR5, and AtBRR6 genes are compared to a wild type Col-0 control.

FIG. 3A depicts a graph of average seed set per silique (seed pod) for Arabidopsis AtBRR1, AtBRR2, AtBRR3, AtBRR4, AtBRR5 and AtBRR6 knockouts versus a wild type Col-0 control. Knockouts in BRR genes did not affect plant fecundity.

FIG. 3B depicts a graph of average seed set per silique (seed pod) for Arabidopsis BRR knockouts versus Col-0 control, including first generation (F1) progeny that are heterozygous for both genes listed. Knockouts in BRR genes did not affect plant fecundity.

FIG. 4A and FIG. 4B depict graphical results for peroxidase (PDX) assay for two tomato lines (Solanum lycopersicum) carrying premature stop codons in BRR genes. In FIG. 4A, a Q240* knockout in SIBRR5b displayed an elevated PDX response, but was not significantly different (n=12 plants per genotype, p=0.07). In FIG. 4B, a Q581* knockout in SIBRR4b displayed a significantly elevated PDX response (n=6-9 plants per genotype, p=0.03).

FIG. 5A depicts a graph of bacterial density (cfu/cm²) for a tomato plant (Solanum lycopersicum) with a Q240* SIBRR5b knockout challenged with Pseudomonas syringae DC3000. Bacterial colonization was measured 3 days post inoculation. The plant with the knockout of the SIBRR5b gene (black bar, “aa”) had significantly lower bacterial accumulation than wild type sibling controls (grey, “AA”).

FIG. 5B depicts a graph of bacterial density (cfu/cm²) for a tomato plant (Solanum lycopersicum) with a W807* SIBRR6 knockout challenged with Pseudomonas syringae DC3000. Bacterial colonization was measured 3 days post inoculation. The plant with the knockout of the SIBRR6 gene (black bar, “aa”) had significantly lower bacterial accumulation than wild type sibling controls (grey, “AA”).

FIG. 5C depicts a graph of bacterial growth in tomato plants (Solanum lycopersicum) challenged with Pseudomonas syringae DC3000. Bacterial colonization was measured 3 days post inoculation. Data is normalized and shown as fold growth compared to the average bacterial load of the matched wild type control. The legend at the right of the graph read from top to bottom correspond to the bars in the graph read from left to right.

FIG. 6A depicts a graph of disease severity score vs. days post inoculation for tomato plants (Solanum lycopersicum) challenged with Xanthomonas gardneri. Plants were inoculated with a bacterial suspension and then scored for disease severity over a 12-day time course (n=9-10 plants of each genotype). The light gray bars are for the vendor wild type plants, the dark gray bars are for a SIBRR6 Q388* knockout and the black bars are for wild type sibling controls. The SIBRR6 Q388* knockout (‘aa’) displays greater resistance to the Xanthomonas gardneri challenge than the vendor wild type and the wild type sibling controls.

FIG. 6B depicts a graph of area under a disease progression (AUDPC) curve derived from FIG. 6A, calculated for the 12-day time course, confirming that the SIBRR6 Q388* knockout (‘aa’) displays greater resistance to the Xanthomonas gardneri challenge than the wild type sibling controls (‘AA”).

### DETAILED DESCRIPTION

The present methods involve very closely related genes and proteins encoded by the genes, which when non-functional in the plant confer broad-range resistance to plants against pathogens, particularly against important agricultural pathogens. These genes and their corresponding proteins are called Broad-Range Resistance (BRR) genes and proteins. The BRR genes are part of the same clade of genes in a larger class of genes that encode leucine-rich repeat, receptor-like kinases (LRR-RLKs). LRR-LRKs are generally involved in pathogen detection or developmental processes. Normally the loss of functioning of an immune-related LRR-RLK dampens the immune response, and is therefore detrimental to plant disease resistance. Further, loss of functioning of an immune-related LRR-RLK is often detrimental to developmental processes in the plant. Unexpectedly, loss of functioning in a plant of a particular group of BRR genes or the proteins encoded by the genes results in increased immune response in the plant without unduly affecting plant development.

The group of BRR genes belong to a closely related clade of genes within the larger LRR-RLK class. The BRR proteins are rich in leucine, the amino acid sequences of the BRR proteins having an amount of leucine amino acids of at least about 10%, preferably at least about 12%, based on the total number of amino acids in the sequence. The amount of leucine in the BRR proteins is preferably about 25% or less, or about 20% or less or about 16% or less. In one embodiment, the amount of leucine in the BRR proteins is in a range of about 12.5% to about 15.6%.

In one embodiment, the BRR genes and proteins of interest are those from Arabidopsis thaliana listed in Table 1 and those BRR genes and proteins from other plant species which are homologous to the A. thaliana BRR genes and proteins. Homologs are genes or proteins in different species, which are derived from a common ancestor. Homologs may comprise orthologs and/or paralogs. Orthologs are genes or proteins in different species that evolved from a common ancestral gene by speciation. Normally, orthologs retain the same function in the course of evolution. Paralogs are genes related by duplication within a genome. Each paralog is co-orthologous to the unduplicated gene in Arabidopsis. The amino acid sequence of the LRR-RLK polypeptide in A. thaliana may be the amino acid sequence as set forth in SEQ ID NO: 6, SEQ ID NO: 8, SEQ ID NO: 10 or SEQ ID NO: 12.

TABLE 1

| | | | Nucleotide | Polypeptide |
|---|---|---|---|---|
| Gene ID | Gene Name | BRR # | Sequence | Sequence |
| AT3G51740 | IMK2 | AtBRR3 | SEQ ID NO: 5 | SEQ ID NO: 6 |
| AT3G56100 | MRLK | AtBRR4 | SEQ ID NO: 7 | SEQ ID NO: 8 |
| AT4G20940 | GHR1 | AtBRR5 | SEQ ID NO: 9 | SEQ ID NO: 10 |
| AT5G10020 | SIRK1 | AtBRR6 | SEQ ID NO: 11 | SEQ ID NO: 12 |

The BRR gene and/or protein of other plant species which are useful in the methods of the present invention may have a percentage identity with the bases of the homologous A. thaliana nucleotide sequence, or the amino acids of the homologous A. thaliana polypeptide sequence, of at least about: 50%, 51%, 52%, 53%, 54%, 55%, 56%, 57%, 58%, 59%, 60%, 61%, 62%, 63%, 64%, 65%, 66%, 67%, 68%, 69%, 70%, 71%, or 72%. Alternatively, or additionally, the BRR protein of other plant species may have a percentage similarity with the amino acids of the homologous A. thaliana polypeptide sequence of at least about: 70%, 71%, 72%, 73%, 74%, 75%, 76%, 77%, 78%, 79% or 80%. Whether a gene and/or protein in a plant is homologous to a BRR gene and/or protein in Arabidopsis thaliana may be determined by standard methods in the fields of bioinformatics and comparative genomics, which are well known to one skilled in the art.

In the context of the present disclosure, NCBI BLAST may be used to identify candidate BRR genes or proteins from a target species. For example, BLASTP may be performed against a restricted database of protein sequences from the target species. All positive candidates may be identified as those high scoring segment pairs (HSPs, i.e. BLAST ‘hits’) that fulfill the following criteria: have an expect threshold of less than or equal to 1.0; and contains both protein kinase and leucine-rich extracellular conserved domains. The identified candidate sequences may be combined with the Arabidopsis thaliana BRR protein sequences into a single dataset. A global multiple sequence alignment may be performed on the dataset with a state-of-the-art alignment program that incorporates both an affine gap model and iterative realignment (e.g. MUSCLE, MAFFT). A phylogenetic analysis may be performed on the alignment using a program that incorporates state-of-the-art Maximum Likelihood or Bayesian phylogenetic methodologies. The most inclusive clade that contains all of the A. thaliana protein sequences and at least one protein sequence from the target organism may be identified. The clade may be supported by a standard phylogenetic support test (e.g. bootstrap score greater than or equal to 50%, or Bayesian posterior probability greater than or equal to 80%). A clade is a phylogenetic cluster that contains all sequences that descent from a common ancestral sequence. An ancestral sequence is an interior node in the phylogenetic tree. All proteins within the clade defined above can be considered candidate BRR proteins, and the corresponding genes can be considered BRR genes. Additionally, any protein sequence from the target species containing both protein kinase and leucine-rich extracellular conserved domains identified as a ‘top hit’ HSP by BLASTP can be included. All A. thaliana BRRs may be used as the BLASTP query against a restricted database of protein sequences from the target species and the highest scoring HSP for each BRR (HSPs are ranked from best to worst based on low E-values, and high query coverage and percent identity) may be identified and retained. Multiple protein sequences may be retained in the case that there is a tie for ‘top hit’ HSP, which may also be considered as BRR candidates.

In the present methods, to identify or obtain plants possessing broad-range resistance against pathogens, screening techniques, preferably high-throughput screening techniques, may be performed to identify plant genes that act as immune receptors or co-receptors that recognize or respond to conserved microbial features (so called microbe associated molecular patterns (MAMPs)). The recognition of MAMPs by immune receptors elicits an innate immune response that protects the host from infectious diseases. Knocking out or reducing the function of the identified plant genes, attenuating or eliminating the function of the protein encoded by the identified plant genes, identifying mutant plants that already have knocked-out or reduced function of the identified genes or identifying mutant plants that already have attenuated or eliminated function of the protein encoded by the identified plant genes, followed by confirming an increase in immune response or decrease in disease severity of the plant provides plants with increased disease resistance.

In one embodiment, screens, particularly high-throughput screens, may be used to test a large number of mutant plants that lack functioning of a single or more than one known member of the large family of LRR-RLKs for their ability to respond to a suite of MAMPs or their ability to respond to a pathogen challenge. The lack of functioning may arise from single point mutations, or other knockouts of the LRR-RLK gene or genes. For example, the mutated gene in the plant may comprise one or more alternative splicing isoforms, one or more premature stop codons, one or more codons that encode different amino acids, and/or other mutations, leading to a different polypeptide than the functioning LRR-RLK polypeptide. Normally the loss of functioning of an immune-related LRR-RLK dampens immune response, and is therefore detrimental to plant disease resistance. Consequently, the majority of the mutants in the screen may either show reduced or no response to the MAMPs or the pathogen challenge. However, the mutants in which the loss of functioning is related to one or more BRR genes or proteins may show an increase in response, thereby providing a plant with increased disease resistance. For a species of interest, if there is an accessible library of knockout lines, the library of knockout lines may be screened for candidates that lack the known functioning LRR-RLK. If there is no accessible library of knockout lines to screen, other screening techniques may be used on cultivars. For example, RNAseq technology may be used to screen existing cultivars for any that lack members of this group or show reduced expression, which then can be used as molecular markers in traditional breeding programs to produce highly resistant cultivars. RNAseq refers to a group of technologies used to sequence the entire set of messenger RNA (mRNA) molecules, or transcripts, which are expressed at a given time, in a given tissue. Sequence reads are mapped to a reference genome, or assembled de novo, and expression of each transcript is quantified by various software algorithms, such as HTSeq, FeatureCounts, Rcount, Maxcounts, FIXSEQ, Cuffquant, DEseq, and edgeR. Other high throughput methods such as Deep Variant Scanning (DVS) (see, for example, US 2016/047003 and CA 2,911,002, the contents of which are herein incorporated by reference) may be used to identify cultivars carrying desired mutations in specific LRR-RLK genes. DVS is a method to search for genetic variants, or mutations, in target genes of interest through the following steps: 1) PCR amplification of target genes from pooled DNA samples; 2) construction of Illumina sequencing libraries followed by high-throughput sequencing; 3) bioinformatics analysis using k-mers to identify potential mutations in the population; and 4) identification of the line or individual plant carrying the mutation through high-resolution DNA melting.

In another embodiment, plants with increased disease resistance may also be obtained by silencing techniques to create gene knockouts of appropriate BRR genes. Silencing may be accomplished in a number of ways generally known in the art, for example, RNA interference (RNAi) techniques, artificial microRNA techniques, virus-induced gene silencing (VIGS) techniques, antisense techniques, sense co-suppression techniques and targeted mutagenesis techniques.

RNAi techniques involve stable transformation using RNA interference (RNAi) plasmid constructs. Such plasmids are composed of a fragment of the target gene to be silenced in an inverted repeat structure. The inverted repeats are separated by a spacer, often an intron. The RNAi construct driven by a suitable promoter, for example, the Cauliflower mosaic virus (CaMV) 35S promoter, is integrated into the plant genome and subsequent transcription of the transgene leads to an RNA molecule that folds back on itself to form a double-stranded hairpin RNA. This double-stranded RNA structure is recognized by the plant and cut into small RNAs (about 21 nucleotides long) called small interfering RNAs (siRNAs). siRNAs associate with a protein complex (RISC) which goes on to direct degradation of the mRNA for the target gene.

Artificial microRNA (amiRNA} techniques exploit the microRNA (miRNA} pathway that functions to silence endogenous genes in plants and other eukaryotes. In this method, 21 nucleotide long fragments of the gene to be silenced are introduced into a pre-miRNA gene to form a pre-amiRNA construct. The pre-miRNA construct is transferred into the plant genome using transformation methods apparent to one skilled in the art. After transcription of the pre-amiRNA, processing yields amiRNAs that target genes which share nucleotide identity with the 21 nucleotide amiRNA sequence.

Virus-induced gene silencing (VIGS) techniques are a variation of RNAi techniques that exploits the endogenous antiviral defenses of plants. Infection of plants with recombinant VIGS viruses containing fragments of host DNA leads to posttranscriptional gene silencing for the target gene. In one embodiment, a tobacco rattle virus (TRV) based VIGS system can be used.

Antisense techniques involve introducing into a plant an antisense oligonucleotide that will bind to the messenger RNA (mRNA) produced by the gene of interest. The “antisense” oligonucleotide has a base sequence complementary to the gene's messenger RNA (mRNA), which is called the “sense” sequence. Activity of the sense segment of the mRNA is blocked by the anti-sense mRNA segment, thereby effectively inactivating gene expression.

Sense co-suppression techniques involve introducing a highly expressed sense transgene into a plant resulting in reduced expression of both the transgene and the endogenous gene. The effect depends on sequence identity between transgene and endogenous gene.

Targeted mutagenesis techniques, for example TILLING (Targeting Induced Local Lesions IN Genomes) and “delete-a-gene” using fast-neutron bombardment, may be used to knockout gene function in a plant. TILLING involves treating seeds or individual cells with a mutagen to cause point mutations that are then discovered in genes of interest using a sensitive method for single-nucleotide mutation detection. Detection of desired mutations (e.g. mutations resulting in the inactivation of the gene product of interest) may be accomplished, for example, by PCR methods. For example, oligonucleotide primers derived from the gene of interest may be prepared and PCR may be used to amplify regions of the gene of interest from plants in the mutagenized population. Amplified mutant genes may be annealed to wild-type genes to find mismatches between the mutant genes and wild-type genes. Detected differences may be traced back to the plants which had the mutant gene thereby revealing which mutagenized plants will have the desired expression (e.g. silencing of the gene of interest). These plants may then be selectively bred to produce a population having the desired expression. TILLING can provide an allelic series that includes missense and knockout mutations, which exhibit reduced expression of the targeted gene. TILLING is touted as a possible approach to gene knockout that does not involve introduction of transgenes, and therefore may be more acceptable to consumers. Fast-neutron bombardment induces mutations, i.e. deletions, in plant genomes that can also be detected using PCR in a manner similar to TILLING.

Functional redundancy in BRR genes or the proteins encoded by the genes may lead to the desire to obtain plants in which the loss of functioning is related to more than one BRR gene or protein. Reducing or eliminating the functioning of more than one BRR gene or protein helps prevent one gene or protein from compensating for the loss of functioning of another gene or protein. For example, plants having a loss of functioning of any two, three or all four of BRR3, BRR4, BRR5 and BRR6 may be desirable.

The present methods comprise confirming that the plant has an increased immune response or decreased disease severity upon pathogen challenge compared to the wild type plant. Confirming the phenotype may be accomplished using any one or more of a variety of techniques. In one embodiment, chemical or biological assays may be used to measure the immune response or disease severity, and the measured immune response or disease severity compared to the immune response or disease severity of the wild type plant measured using the same assay. In another embodiment, the target BRR protein may be identified as homologous to a BRR protein in another species of plant, where attenuation or elimination of the activity of the BRR protein in the other species of plant is known to increase immune response or disease severity, thereby providing confirmation that attenuation or elimination of the target BRR protein would also result in increased immune response or decreased disease severity. In yet another embodiment, the plant in which the activity of the target BRR protein has been attenuated or eliminated, or a population of such plants, may be observed over time to determine whether or how the plant or population of plants is affected by a disease.

Chemical or biological assays useful for confirming an increased immune response or a decreased disease severity upon pathogen challenge may involve challenging the plant or plant cells with a compound (e.g. a small molecule or an antigen) or a microbe (e.g. a bacterium) to elicit an immune response in the plant or plant cell, and then measuring the extent of the response or disease severity in comparison to the wild type plant or plant cell. The extent of immune response or disease severity may be measured by the production of certain chemical compounds (e.g. oxidation products, anti-microbial compounds), the expression of pathogenesis-related genes, or the occurrence of other phenotypic changes (e.g. cell death or plant death). Some examples of known chemical and biological assays include: a peroxidase assay involving eliciting an immune response with a flg22 peptide from Pseudomonas aeruginosa; a bacterial growth assay involving challenging the plant with Pseudomonas tomato DC3000; an oomycete growth assay involving infecting the plant with Hyaloperonospora arabidopsidis isolate Noco2; and, assays measuring disease severity in response to infection with pathogens such as Oidium lycopersicum, Leveillula taurica, Xanthomonas spp., Clavibacter michiganensis, Pythium aphanidermatum or Botrytis cinerea.

The disease may be caused by a pathogenic microorganism, for example bacteria, fungi, water molds (Oomycetes) and viruses. Some examples of pathogenic bacteria include Clavibacter michiganensis, Erwinia spp., Agrobacterium spp., Burkholderia spp., Xanthomonas spp., Pseudomonas spp. (e.g. Pseudomonas syringae pv. tomato), Candidatus Phytoplasma and Spiroplasma. Some examples of pathogenic viruses include Tobacco mosaic virus, Tobacco ringspot virus, Tobacco rattle virus, Beet necrotic yellow vein virus, Bean common mosaic virus, Pepper Mild Mottle Virus, Cauliflower mosaic virus and Pepino Mosaic Virus. Some examples of pathogenic fungi include fungi of the Ascomycetes family including Fusarium spp., Thielaviopsis spp., Verticillium spp., Oidium lycopersicum, Leveillula taurica, Botrytis cinerea, Magnaporthe grisea and Sclerotinia sclerotiorum, and fungi of the Basidiomycetes family including Ustilago spp., Rhizoctonia spp., Phakospora pachyrhizi, Puccinia spp. and Armillaria spp. Some examples of pathogenic oomycetes include Pythium spp. and Phytopthora spp.

The method may be used to increase disease resistance in any plant. Crop plants are particularly preferred. Some examples of plants include plants from the following families: Apiaceae, Asteraceae, Brassicaceae, Chenopodiaceae, Cucurbitaceae, Ericaceae, Fabaceae, Lamiaceae, Liliaceae, Poaceae, Polygonaceae, Rosaceae and Solanaceae.

Some specific examples of plants include pepper (bell and chili), tomato, potato, eggplant, tobacco, tomatillo, horseradish, cabbage, cauliflower, broccoli, kohlrabi, kale, Brussels sprout, turnip, Chinese cabbage, radish, rapeseed, mustard, collard, watercress, pak choi, bok choi, rutabaga, cucumber, melons, watermelon, summer squash, pumpkin, gourd, winter squash, apple, peach, apricot, nectarine, plum, strawberry, blackberry, raspberry, pear, cherry, quince, almond, bean, pea, lentil, peanut, soybean, edamame, garbanzo bean, fava bean, hairy vetch, vetches, alfalfa, clover, cowpea, birdsfoot trefoil, black medic, corn, wheat, barley, oat, sorghum, rice, millet, rye, ryegrass, sorghum-sudangrass, fescue, timothy, buckwheat, rhubarb, asparagus, onion, leek, chive, garlic, shallot, lavender, basil, marjoram, oregano, rosemary, sage, thyme, mint, catnip, blueberry, cranberry, spinach, beet, chard, sugar beet, carrot, parsnip, celery, dill, chervil, cilantro, parsley, caraway, fennel, sunflower, lettuce, endive, escarole, radicchio, dandelion, Jerusalem artichoke, artichoke, safflower, chicory, tarragon, chamomile and echinacea.

In some embodiment, the LRR-RLK polynucleotide in the disease resistant plant may include, for example: a polynucleotide comprising the polynucleotide as set forth in SEQ ID NO: 17, wherein a codon of SEQ ID NO: 17 encoding glutamine at position 240 of SEQ ID NO: 18 is replaced with a stop codon; a polynucleotide comprising the polynucleotide as set forth in SEQ ID NO: 17, wherein a codon of SEQ ID NO: 17 encoding glutamine at position 581 of SEQ ID NO: 18 is replaced with a stop codon; a polynucleotide comprising the polynucleotide as set forth in SEQ ID NO: 23, wherein a codon of SEQ ID NO: 23 encoding glutamine at position 388 of SEQ ID NO: 24 is replaced with a stop codon; a polynucleotide comprising the polynucleotide as set forth in SEQ ID NO: 23, wherein a codon of SEQ ID NO: 23 encoding tryptophan at position 807 of SEQ ID NO: 24 is replaced with a stop codon; a polynucleotide comprising the polynucleotide as set forth in SEQ ID NO: 23, wherein codons of SEQ ID NO: 23 encoding glutamine at position 388 and tryptophan at position 807 of SEQ ID NO: 24 are replaced with stop codons; a polynucleotide comprising the polynucleotide as set forth in SEQ ID NO: 35 and further comprising a splice site disruption; a polynucleotide comprising the polynucleotide as set forth in SEQ ID NO: 33, wherein a codon of SEQ ID NO: 33 encoding glutamine at position 465 of SEQ ID NO: 34 is replaced with a stop codon; a polynucleotide comprising the polynucleotide as set forth in SEQ ID NO: 37, wherein a codon of SEQ ID NO: 37 encoding glutamine at position 961 of SEQ ID NO: 38 is replaced with a stop codon; and, a polynucleotide comprising the polynucleotide as set forth in SEQ ID NO: 39, wherein a codon of SEQ ID NO: 39 encoding glutamine at position 915 of SEQ ID NO: 40 is replaced with a stop codon.

### Examples

### Materials and Methods:

### Initial Screen

T-DNA insertion knockout lines were screened with a peroxidase assay for 169 genes out of 227 candidate immune receptors identified in the Arabidopsis thaliana genome. Knockout lines were challenged with 6 novel microbe-associated molecular patterns (MAMPS) from Pseudomonas syringae, as well as positive and negative control peptides. A high-throughput peroxidase assay, with six replicate plant/genotype and ten leaf cores per plant, was used as a read out of the oxidative burst associated with pattern-triggered immunity. This screen identified a group of related receptors in a single clade that show a generalized increase in immunity when knocked out. The related receptors are called Broad-Range Resistance (BRR) receptors.

### Deep Variant Scanning

Target genes of interest in tomato were amplified by PCR from pooled DNA samples representing mutagenized M2 plants from a tomato population. Amplicons representing multiple genes were pooled stoichiometrically and prepared for high-throughput Illumina sequencing with a Nextera™ XT kit. The Deep Variant Scanning (DVS) bioinformatics pipeline was used to call likely single nucleotide polymorphisms (SNPs, i.e. mutations) relative to the wild type sequence. Putative mutations were confirmed and assigned to an individual M2 family by High-Resolution DNA melting. The DVS method is more fully described in Canadian patent 2,911,0002.

### Peroxidase Assay

Single leaves were excised from six plants per genotype of mature plants. From each leaf, a series of size one leaf cores were taken and washed for 1 hour in 1 ml of 1× monobasic (MS) solution with agitation. After washing, leaves were transferred to individual wells of a clear 96-well assay plate avoiding the use of the edge wells to minimize evaporation effects. Each well received 50 μl of 1×MS buffer alone, or supplemented with 1 μM of flg22 peptide. The flg22 peptide is a peptide with its sequence derived from the flagellin N-terminus of Pseudomonas aeruginosa and is known to elicit specific innate immune response in plants as well as animals. Plates were sealed with parafilm and incubated for 20 hours with agitation. The leaf disks were removed and each well received 50 μL of a 1 mg/ml solution of 5-aminosalicylic acid (A79809, Sigma-Aldrich) pH 6.0 with 0.01% hydrogen peroxide. The reaction was allowed to proceed for 1-3 minutes and stopped by the addition of 20 μM 2N NaOH prior to reading the OD600 on a POLARstar OPTIMA™ microplate reader (BMG Labtech).

For tomato (Solanum lycopersicum), after the initial wash the disks were vacuum infiltrated with the flg22 peptide for 10 minutes at 90 kPa. The disks were then incubated for 1 hour with agitation. The buffer was removed and replaced with fresh 1×MS prior to the 20-hour incubation.

### Bacterial Growth Assay

A. thaliana or tomato (Solanum lycopersicum) plants were pressure infiltrated with Pseudomonas tomato DC3000 (0.0005 OD600). Bacterial growth was measured after 24 hours for Arabidopsis and 72 hours for tomato. Briefly, surface sterilized leaf disks were homogenized and the resulting culture was serially diluted and plated on solid selective media. The resulting colony counts were used to determine the number of colony forming units per surface area of the leaf. Each treatment was conducted on 4 leaves per plant for Arabidopsis and 2 leaves per plant for tomato of 6 individual plants.

### Oomycete Growth Assay

Infection with Hyaloperonospora arabidopsidis isolate Noco2 was performed by applying a single drop of asexual inoculum suspension (106 conidiosporangia/mL) per cotyledon of 7-d-old A. thaliana seedlings. The seedlings were grown at 16° C. and >90% RH with an 8-h photoperiod. The total number of sporangiophores per cotyledon was counted at 7 d after inoculation.

### Seed Set Quantification

Four individual plants of each A. thaliana genotype were allowed to mature and form siliques. From each plant two random siliques were harvested and the fully-formed seeds were counted. The experiment was performed twice, showing similar results.

Xanthomonas gardneri Inoculation

A two-day old culture of Xanthomonas gardneri (Isolate Xg DC 00T7A), grown on NBY agar was used to inoculate a liquid culture of sucrose peptone media. The culture was grown in a flask in a rotary shaker (210 RPM, 28° C.) for 24 hours. The culture was then centrifuged, resuspended in autoclaved tap water, and adjusted to an OD600 of 0.3 using a spectrophotometer. Silwet™ L-77 0.05% (v/v) was added to the suspension before the inoculum was transferred to a spray bottle. Four-week old tomato plants were preconditioned in a misting chamber for 4 hours prior to inoculation. The inoculum was applied to both sides of tomato leaves, making sure to cover the underside. After inoculation, the misting frequency was adjusted to 10 seconds of mist every 10 minutes. Plants were kept in the humid/mist chamber for 24 hours at 21-23° C. Following 24 hours in the mist chamber, plants were removed, and placed on an exposed bench in the greenhouse. When leaves are dry, youngest inoculated leaves were marked with a fluorescent coloured dye (#1162A Luminous Powder Kit—BioQuip Products Inc.) This ensured that disease assessments only include those leaves that were inoculated with pathogen. The rating scaled used was as follows:

0=Symptomless

1=Few necrotic spots on a few leaflets

2=A few necrotic spots on many leaflets

3=Many spots with coalescence on few leaflets

4=Many spots with coalescence on many leaflets

5=Severe disease and leaf defoliation

6=Dead plant

### Results and Discussion:

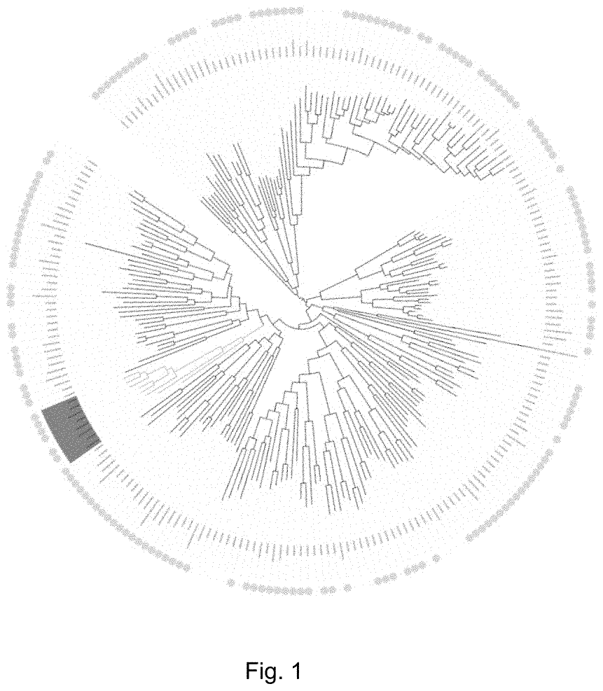

With reference to FIG. 1, a striking feature of the subset of Broad-Range Resistance (BRR) genes is that the highly resistant mutants with BRR gene knockouts are evolutionarily very closely related. This finding suggests that a family of negative regulators of plant immunity has been found, the loss of any one of which results in increased pathogen resistance.

An initial screen of the Arabidopsis thaliana genome revealed six Broad-Range Resistance genes and corresponding proteins, as listed in Table 2. Table 3 provides values from a heat map illustrating that certain knockouts of BRR genes of A. thaliana display elevated responses to multiple immune elicitors in the in vitro peroxidase assay. MAMPs 1-6 represent Pseudomonas syringae MAMPs and flg22 is included as a classical inducer of MAMP-triggered immunity. Values given in Table 3 represent responses as % of non-treated control. Knockouts of AtBRR3, AtBRR4, AtBRR5, and AtBRR6 demonstrate a consistent and generalized increase in immune response. Knockouts of AtBRR1 and AtBRR2 do not display a consistent elevation in immune response.

TABLE 2

| | | | Nucleotide | Polypeptide |
|---|---|---|---|---|
| Gene ID | Gene Name | BRR # | Sequence | Sequence |
| AT2G27060 | — | AtBRR1 | SEQ ID NO: 1 | SEQ ID NO: 2 |
| AT3G24660 | TMKL1 | AtBRR2 | SEQ ID NO: 3 | SEQ ID NO: 4 |
| AT3G51740 | IMK2 | AtBRR3 | SEQ ID NO: 5 | SEQ ID NO: 6 |
| AT3G56100 | MRLK | AtBRR4 | SEQ ID NO: 7 | SEQ ID NO: 8 |
| AT4G20940 | GHR1 | AtBRR5 | SEQ ID NO: 9 | SEQ ID NO: 10 |
| AT5G10020 | SIRK1 | AtBRR6 | SEQ ID NO: 11 | SEQ ID NO: 12 |

TABLE 3

| | MAMP1 | MAMP2 | MAMP3 | MAMP4 | MAMP5 | MAMP6 | flg22 |
|---|---|---|---|---|---|---|---|
| AtBRR1 | 123 | 106 | 102 | 129 | 115 | 97 | 106 |
| AtBRR2 | 115 | 108 | 115 | 111 | 115 | 98 | 97 |
| AtBRR3 | 123 | 109 | 105 | 133 | 118 | 105 | 119 |
| AtBRR4 | 131 | 115 | 121 | 154 | 126 | 104 | 129 |
| AtBRR5 | 139 | 144 | 145 | 152 | 135 | 143 | 156 |
| AtBRR6 | 122 | 112 | 136 | 143 | 138 | 119 | 144 |

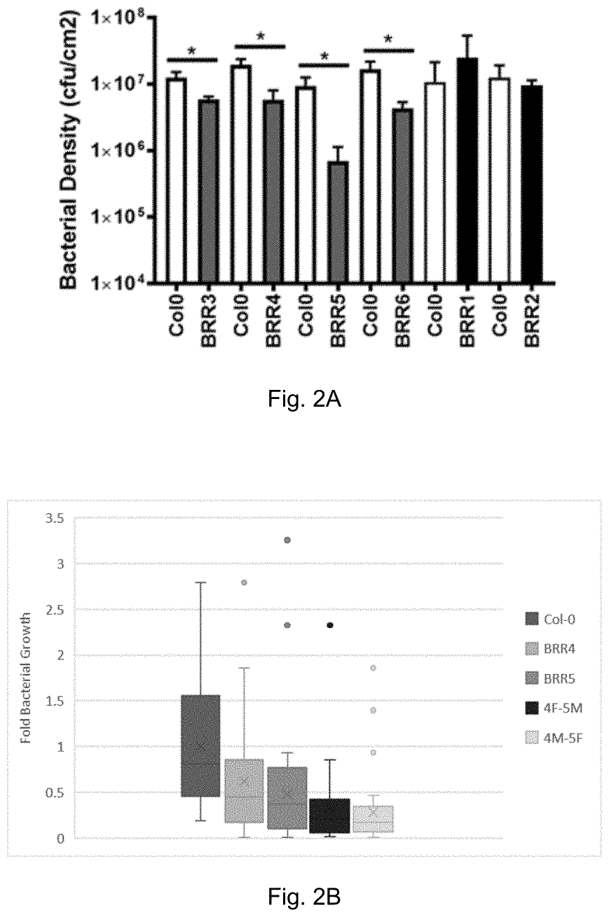

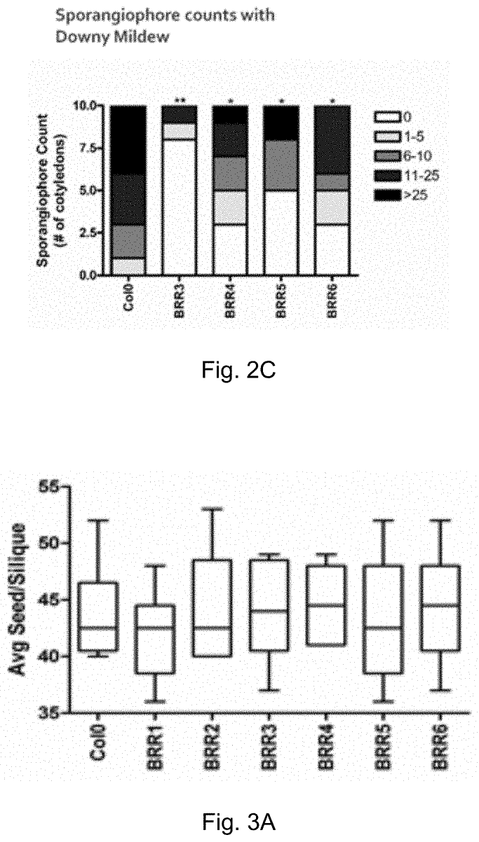

In vivo assays were conducted to more fully evaluate the disease resistance of A. thaliana plants whose genomes contain knockouts of the AtBRR3, AtBRR4, AtBRR5, and AtBRR6 genes. As seen in FIG. 2A, challenging A. thaliana plants with Pseudomonas syringae DC3000, a known plant bacterial pathogen, illustrates that plants having knockouts of the AtBRR3, AtBRR4, AtBRR5, and AtBRR6 genes were significantly more resistant to accumulation of the pathogen than the wild type control plants (Col-0). FIG. 2A further includes test data for A. thaliana plants whose genomes contain knockouts of the AtBRR1 and AtBRR2 genes. Knockouts in AtBRR3, AtBRR4, AtBRR5, and AtBRR6 genes also had significantly lower bacterial accumulation than knockouts in AtBRR1 and AtBRR2 genes, illustrating that confirmation of increased immune response is desirable for plants with mutated or knocked out BRR genes. As further seen in FIG. 2B, while single T-DNA knockouts in AtBRR genes had significantly lower bacterial accumulation than wild type Col-0 controls, first generation (F1) progeny that are heterozygous in both loci also show reduced bacterial growth, which trends towards greater reduction than the single mutant knockouts. 4F-5M indicates that the female of the cross is AtBRR4 and the male of the cross is AtBRR5, while the reverse is true for 4M-5F.

As seen in FIG. 2C, challenging A. thaliana plants with H. arabidopsidis, a plant bacterial pathogen responsible for Downy Mildew, shows that plants having knockouts of the AtBRR3, AtBRR4, AtBRR5, and AtBRR6 genes had significantly reduced sporangiophore count on the cotyledons compared to the wild type control plants (Col-0). The results illustrate that knocking out the AtBRR3, AtBRR4, AtBRR5, and AtBRR6 genes provides plants with a generalized pathogen resistance.

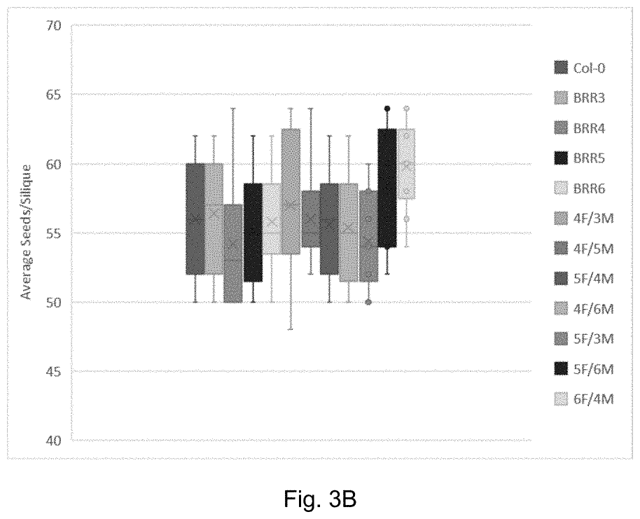

While increased pathogen resistance is desirable, the plants must still have substantially normal development for the increased pathogen resistance to be useful on an agricultural scale. To examine development of the AtBRR3, AtBRR4, AtBRR5, and AtBRR6 A. thaliana knockout plants, seed set qualification experiments were performed to determine fecundity of the plants in comparison to the wild type A. thaliana plant. Four individual plants of each A. thaliana genotype (AtBRR3, AtBRR4, AtBRR5, and AtBRR6 knockouts and wild type Col-0) were allowed to mature and form siliques. From each plant two random siliques were harvested and the fully-formed seeds were counted. As seen in FIG. 3A, knocking out the AtBRR3, AtBRR4, AtBRR5, and AtBRR6 genes in the plants did not affect plant fecundity in comparison to the wild type Col-0 control. The experiment was performed twice, showing similar results. As further seen in FIG. 3B, fecundity of first generation (F1) progeny that are heterozygous for both of the listed genes is also not affected by knocking out the BRR genes. 4F/3M means the female of the cross is AtBRR4 and the male of the cross is AtBRR3. The other F1 designations follow the same pattern of identification.

To extend the methods to other species of plants, the genomes of cultivated populations of tomato (Solanum lycopersicum), pepper (Capsicum annuum) and cucumber (Cucumis sativa) were examined to determine whether they possess BRR genes. EMS-mutagenized populations were then screened using Deep Variant Scanning (DVS) to uncover mutant plants having a non-functional BRR gene. BRR genes and the proteins encoded by the genes were found in all three species as listed in Table 4.

TABLE 4

| | | | |
|---|---|---|---|
| | | Nucleotide | Polypeptide |
| Gene ID | BRR # | Sequence | Sequence |

Tomato (Solanum lycopersicum)

| | | | |
|---|---|---|---|
| Solyc09g015170.2.1 | SIBRR3 | SEQ ID NO: 13 | SEQ ID NO: 14 |
| Solyc05g052350.2.1 | SIBRR4a | SEQ ID NO: 15 | SEQ ID NO: 16 |
| Solyc06g050560.2.1 | SIBRR4b | SEQ ID NO: 17 | SEQ ID NO: 18 |
| Solyc02g023950.2.1 | SIBRR5a | SEQ ID NO: 19 | SEQ ID NO: 20 |
| Solyc02g070000.2.1 | SIBRR5b | SEQ ID NO: 21 | SEQ ID NO: 22 |
| Solyc04g054200.2.1 | SIBRR6 | SEQ ID NO: 23 | SEQ ID NO: 24 |

Pepper (Capsicum annuum)

| | | | |
|---|---|---|---|
| CA07g20500 | CaBRR3 | SEQ ID NO: 25 | SEQ ID NO: 26 |
| CA05g15550 | CaBRR4 | SEQ ID NO: 27 | SEQ ID NO: 28 |
| CA02g04850 | CaBRR5 | SEQ ID NO: 29 | SEQ ID NO: 30 |
| CA12g12250 | CaBRR6 | SEQ ID NO: 31 | SEQ ID NO: 32 |

Cucumber (Cucumis sativa)

| | | | |
|---|---|---|---|
| Cucsa176710.1/ | CsBRR3 | SEQ ID NO: 33 | SEQ ID NO: 34 |
| Csa3M730860.1 | | | |
| Cucsa144260.1/ | CsBRR4 | SEQ ID NO: 35 | SEQ ID NO: 36 |
| Csa5M218200.1 | | | |
| Cucsa.167060.1/ | CsBRR5 | SEQ ID NO: 37 | SEQ ID NO: 38 |
| Csa1M538180.1 | | | |
| Cucsa104840.1/ | CsBRR6 | SEQ ID NO: 39 | SEQ ID NO: 40 |
| Csa6M501870.1 | | | |

In tomato, pepper and cucumber, a number of EMS-induced mutations in BRR genes have been discovered as listed in Table 5, which may lead to knocking out one of the BRR genes in the respective mutant lines. The mutations involve premature stop codons, other single point mutations (SNPs) and splice site (SS) disruptions.

TABLE 5

| | |
|---|---|
| Gene | Mutation |

Tomato

| | |
|---|---|
| SIBRR3 | SS |
| | W101* |
| SIBRR4a | R426* |
| | Q11* |
| SIBRR4b | Q581* |
| SIBRR5a | P734S |
| | G307E |
| SIBRR5b | Q240* |
| SIBRR6 | Q388* |
| | QW807* |

Pepper

| | |
|---|---|
| CaBRR3 | W748* |
| | G617E |
| CaBRR4 | W17* |
| | Q114* |
| CaBRR5 | P128L |
| | P127L |
| | G156S |
| CaBRR6 | M1I |
| | Q389* |

Cucumber

| | |
|---|---|
| CsBRR3 | Q465* |
| | L689F |
| CsBRR4 | SS |
| | L151F |
| | G166D |
| | T200I |
| | L284F |
| | L300F |
| CsBRR5 | Q961* |
| | C927Y |
| | R873K |
| CsBRR6 | S560L |
| | G424E |
| | E864K |
| | Q915* |
| | G960D |

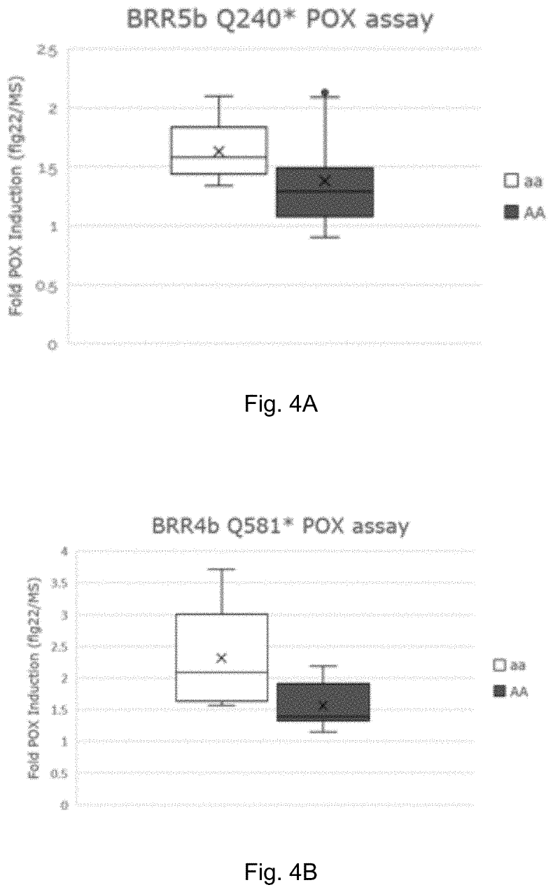

Two of the tomato mutant lines (Q240* and Q581*) were assayed with the peroxidase assay to confirm that the mutant plants exhibit increased immune response. As shown in FIG. 4A, the Q240* knockout in SIBRR5b displayed an elevated PDX response, but was not significantly different (n=12 plants per genotype, p=0.07). As seen in FIG. 4B, the Q581* knockout in SIBRR4b displayed a significantly elevated PDX response (n=6-9 plants per genotype, p=0.03).

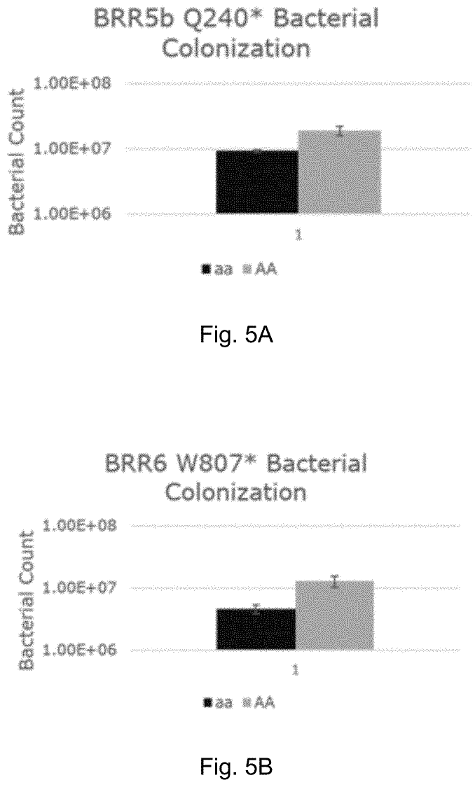

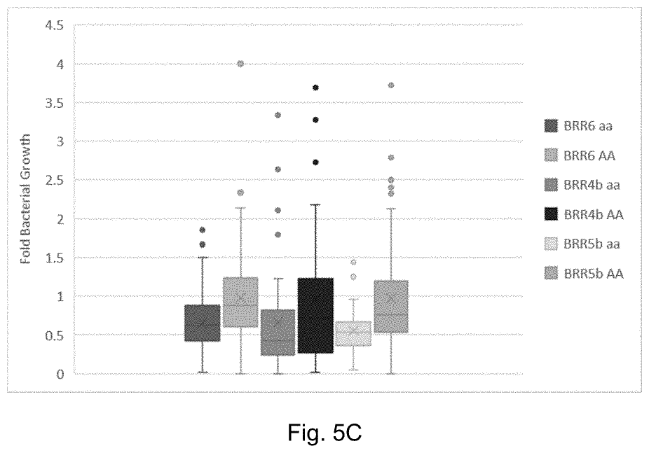

Three of the tomato mutant lines (Q581*, Q240* and W807*) were assayed with the Pseudomonas syringae DC3000 challenge to confirm that the mutant plants exhibit increased resistance to bacterial infection. As shown in FIG. 5A, the Q240* SIBRR5b knockout challenged with Pseudomonas syringae DC3000 (black bar, “aa”) had significantly lower bacterial accumulation than wild type sibling controls (grey, “AA”). As shown in FIG. 5B, the W807* SIBRR6 knockout challenged with Pseudomonas syringae DC3000 (black bar, “aa”) also had significantly lower bacterial accumulation than wild type sibling controls (grey, “AA”). As seen in FIG. 5C, the Q581* SIBRR4b knockout challenged with Pseudomonas syringae DC3000 (BRR4b-aa) had significantly lower bacterial accumulation than wild type sibling controls (BRR4b-AA). FIG. 5C also further confirms the results from FIG. 5A and FIG. 5B; thus, knockouts in BRR genes (aa) had lower bacterial accumulation than matched wild type controls (AA).

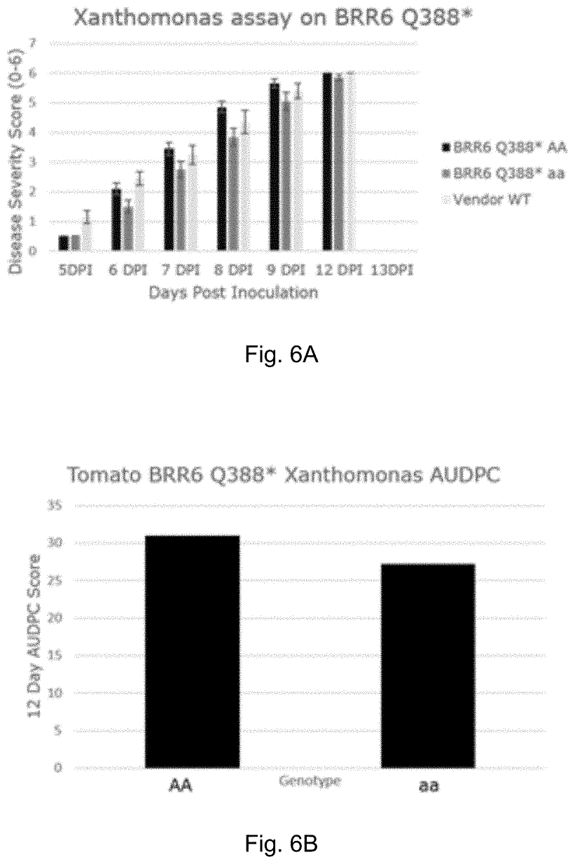

One of the tomato mutant lines (Q388*) was assayed with the Xanthomonas gardneri challenge to confirm that mutant plants also exhibit increased resistance to infection from other bacteria. As shown in FIG. 6A and FIG. 6B, the BRR6 Q388* knockout (dark gray bars in FIG. 6A; ‘aa’ in FIG. 6B) displays greater resistance to the Xanthomonas gardneri challenge than both the vendor wild type (light gray bars in FIG. 6A) and the wild type sibling controls (black bars in FIG. 6A; ‘AA’ in FIG. 6B).

Cumulatively, the results show that the invention can be extended to different species of plants and various plant pathogens.

The novel features will become apparent to those of skill in the art upon examination of the description. It should be understood, however, that the scope of the claims should not be limited by the embodiments, but should be given the broadest interpretation consistent with the wording of the claims and the specification as a whole.

1. A method of obtaining a plant with increased disease resistance, the method comprising:
determining whether a species of plant has a wild type gene that encodes a functioning leucine-rich repeat, receptor-like kinase (LRR-RLK) polypeptide having at least 50% percent sequence identity to the amino acid sequence of a homologous LRR-RLK polypeptide in Arabidopsis thaliana;
identifying a plant having reduced or eliminated functioning of the LRR-RLK polypeptide by in a population of the species, identifying a plant that contains a mutated gene instead of the wild type gene and determining whether the mutated gene in the plant encodes a different polypeptide than the functioning LRR-RLK polypeptide, or
silencing expression of the wild type gene or the functioning LRR-RLK polypeptide; and,
confirming that the plant with the reduced or eliminated functioning of the LRR-RLK polypeptide has an increased immune response or a decreased disease severity compared to a wild type plant, thereby obtaining a plant with increased disease resistance.

2. A method of increasing disease resistance in a plant, the method comprising:
attenuating or eliminating activity in the plant of a functioning leucine-rich repeat, receptor-like kinase (LRR-RLK) polypeptide, or silencing expression in the plant of a functioning polynucleotide encoding the functioning LRR-RLK polypeptide, the functioning LRR-RLK polypeptide having at least 50% percent sequence identity to the amino acid sequence of a homologous LRR-RLK polypeptide in Arabidopsis thaliana; and,
confirming that the plant having the attenuated or eliminated activity or the silenced expression has an increased immune response or a decreased disease severity compared to a wild type plant of the same species, thereby producing a plant with increased disease resistance.

3. The method of claim 1, wherein the silencing comprises the use of RNA interference (RNAi), artificial microRNA, virus-induced gene silencing (VIGS), antisense sequences, sense co-suppression or targeted mutagenesis.

4. The method of claim 1, wherein the amino acid sequence of the a homologous LRR-RLK polypeptide in Arabidopsis thaliana is the amino acid sequence as set forth in SEQ ID NO: 6, SEQ ID NO: 8, SEQ ID NO: 10 or SEQ ID NO: 12.

5. The method of claim 1, wherein the amino acid sequence of the functioning LRR-RLK polypeptide has at least about 10% leucine, based on total number of amino acids in the amino acid sequence.

6. The method of claim 1, further comprising propagating the plant with increased disease resistance to produce a disease resistant population of the plants.

7. The method of claim 1, wherein the confirming that the plant has an increased immune response comprises measuring the immune response in a chemical or biological assay and comparing the measured immune response to an immune response of the wild type plant in the same assay.

8. The method of claim 1, wherein the disease is caused by a microorganism.

9. The method of claim 1, wherein the microorganism is Clavibacter michiganensis, Erwinia spp., Agrobacterium spp., Burkholderia spp., Xanthomonas spp., Pseudomonas spp., Candidatus Phytoplasma, Spiroplasma, Tobacco mosaic virus, Tobacco ringspot virus, Tobacco rattle virus, Beet necrotic yellow vein virus, Bean common mosaic virus, Pepper Mild Mottle Virus, Cauliflower mosaic virus, Pepino Mosaic Virus, Fusarium spp., Thielaviopsis spp., Verticillium spp., Oidium lycopersicum, Leveillula taurica, Botrytis cinerea, Magnaporthe grisea, Sclerotinia sclerotiorum, Ustilago spp., Rhizoctonia spp., Phakospora pachyrhizi, Puccinia spp., Armillaria spp., Pythium spp. or Phytopthora spp.

10. The method of claim 1, wherein the microorganism is Clavibacter michiganensis, Botrytis cinerea, Oidium lycopersicum, Leveillula taurica, Pseudomonas syringae, Hyaloperonospora arabidopsidis, Pepino Mosaic Virus, Phytopthora spp., or Pythium spp.

11. The method of claim 1, wherein the plant is pepper, tomato, potato, eggplant, tobacco, tomatillo, horseradish, cabbage, cauliflower, broccoli, kohlrabi, kale, Brussels sprout, turnip, Chinese cabbage, radish, rapeseed, mustard, collard, watercress, pak choi, bok choi, rutabaga, cucumber, melons, watermelon, summer squash, pumpkin, gourd, winter squash, apple, peach, apricot, nectarine, plum, strawberry, blackberry, raspberry, pear, cherry, quince, almond, bean, pea, lentil, peanut, soybean, edamame, garbanzo bean, fava bean, hairy vetch, vetches, alfalfa, clover, cowpea, birdsfoot trefoil, black medic, corn, wheat, barley, oat, sorghum, rice, millet, rye, ryegrass, sorghum-sudangrass, fescue, timothy, buckwheat, rhubarb, asparagus, onion, leek, chive, garlic, shallot, lavender, basil, marjoram, oregano, rosemary, sage, thyme, mint, catnip, blueberry, cranberry, spinach, beet, chard, sugar beet, carrot, parsnip, celery, dill, chervil, cilantro, parsley, caraway, fennel, sunflower, lettuce, endive, escarole, radicchio, dandelion, Jerusalem artichoke, artichoke, safflower, chicory, tarragon, chamomile or echinacea.

12. The method of claim 1, wherein the plant is Solanum lycopersicum, Capsicum annuum or Cucumis sativa.

13. A disease resistant plant comprising a leucine-rich repeat, receptor-like kinase (LRR-RLK) polynucleotide having silenced expression of a functioning LRR-RLK polypeptide, the LRR-RLK polypeptide having at least 50% percent sequence identity to the amino acid sequence of a homologous LRR-RLK polypeptide in Arabidopsis thaliana, wherein the plant is pepper, tomato, potato, eggplant, tobacco, tomatillo, horseradish, cabbage, cauliflower, broccoli, kohlrabi, kale, Brussels sprout, turnip, Chinese cabbage, radish, rapeseed, mustard, collard, watercress, pak choi, bok choi, rutabaga, cucumber, melons, watermelon, summer squash, pumpkin, gourd, winter squash, apple, peach, apricot, nectarine, plum, strawberry, blackberry, raspberry, pear, cherry, quince, almond, bean, pea, lentil, peanut, soybean, edamame, garbanzo bean, fava bean, hairy vetch, vetches, alfalfa, clover, cowpea, birdsfoot trefoil, black medic, corn, wheat, barley, oat, sorghum, rice, millet, rye, ryegrass, sorghum-sudangrass, fescue, timothy, buckwheat, rhubarb, asparagus, onion, leek, chive, garlic, shallot, lavender, basil, marjoram, oregano, rosemary, sage, thyme, mint, catnip, blueberry, cranberry, spinach, beet, chard, sugar beet, carrot, parsnip, celery, dill, chervil, cilantro, parsley, caraway, fennel, sunflower, lettuce, endive, escarole, radicchio, dandelion, Jerusalem artichoke, artichoke, safflower, chicory, tarragon, chamomile or Echinacea.

14. The plant of claim 13, wherein the amino acid sequence of the a homologous LRR-RLK polypeptide in Arabidopsis thaliana is the amino acid sequence as set forth in SEQ ID NO: 6, SEQ ID NO: 8, SEQ ID NO: 10 or SEQ ID NO: 12.

15. The plant of claim 13, wherein the LRR-RLK polynucleotide comprises:
the polynucleotide as set forth in SEQ ID NO: 17, wherein a codon of SEQ ID NO: 17 encoding glutamine at position 581 of SEQ ID NO: 18 is replaced with a stop codon;
the polynucleotide as set forth in SEQ ID NO: 21, wherein a codon of SEQ ID NO: 17 encoding glutamine at position 240 of SEQ ID NO: 22 is replaced with a stop codon;
the polynucleotide as set forth in SEQ ID NO: 23, wherein a codon of SEQ ID NO: 23 encoding glutamine at position 388 of SEQ ID NO: 24 is replaced with a stop codon;
the polynucleotide as set forth in SEQ ID NO: 23, wherein a codon of SEQ ID NO: 23 encoding tryptophan at position 807 of SEQ ID NO: 24 is replaced with a stop codon;
the polynucleotide as set forth in SEQ ID NO: 23, wherein codons of SEQ ID NO: 23 encoding glutamine at position 388 and tryptophan at position 807 of SEQ ID NO: 24 are replaced with stop codons;
the polynucleotide as set forth in SEQ ID NO: 35 and further comprising a splice site disruption;
the polynucleotide as set forth in SEQ ID NO: 33, wherein a codon of SEQ ID NO: 33 encoding glutamine at position 465 of SEQ ID NO: 34 is replaced with a stop codon;
the polynucleotide as set forth in SEQ ID NO: 37, wherein a codon of SEQ ID NO: 37 encoding glutamine at position 961 of SEQ ID NO: 38 is replaced with a stop codon; or,
the polynucleotide as set forth in SEQ ID NO: 39, wherein a codon of SEQ ID NO: 39 encoding glutamine at position 915 of SEQ ID NO: 40 is replaced with a stop codon.

16. The plant of claim 13, wherein the LRR-RLK polynucleotide comprises:
the polynucleotide as set forth in SEQ ID NO: 13 and further comprising a splice site disruption;
the polynucleotide as set forth in SEQ ID NO: 13, wherein a codon of SEQ ID NO: 13 encoding tryptophan at position 101 of SEQ ID NO: 14 is replaced with a stop codon;
the polynucleotide as set forth in SEQ ID NO: 15, wherein a codon of SEQ ID NO: 15 encoding arginine at position 426 of SEQ ID NO: 16 is replaced with a stop codon;
the polynucleotide as set forth in SEQ ID NO: 15, wherein a codon of SEQ ID NO: 15 encoding glutamine at position 11 of SEQ ID NO: 16 is replaced with a stop codon;
the polynucleotide as set forth in SEQ ID NO: 17, wherein a codon of SEQ ID NO: 17 encoding glutamine at position 581 of SEQ ID NO: 18 is replaced with a stop codon;
the polynucleotide as set forth in SEQ ID NO: 19, wherein a codon of SEQ ID NO: 19 encoding proline at position 734 of SEQ ID NO: 20 is replaced with serine;
the polynucleotide as set forth in SEQ ID NO: 19, wherein a codon of SEQ ID NO: 19 encoding glycine at position 307 of SEQ ID NO: 20 is replaced with glutamic acid;
the polynucleotide as set forth in SEQ ID NO: 21, wherein a codon of SEQ ID NO: 22 encoding glutamine at position 240 of SEQ ID NO: 22 is replaced with a stop codon;
the polynucleotide as set forth in SEQ ID NO: 23, wherein a codon of SEQ ID NO: 23 encoding glutamine at position 388 of SEQ ID NO: 24 is replaced with a stop codon;
the polynucleotide as set forth in SEQ ID NO: 23, wherein a codon of SEQ ID NO: 23 encoding tryptophan at position 807 of SEQ ID NO: 24 is replaced with a stop codon;
the polynucleotide as set forth in SEQ ID NO: 23, wherein codons of SEQ ID NO: 23 encoding glutamine at position 388 and tryptophan at position 807 of SEQ ID NO: 24 are replaced with stop codons;
the polynucleotide as set forth in SEQ ID NO: 25, wherein a codon of SEQ ID NO: 25 encoding tryptophan at position 748 of SEQ ID NO: 26 is replaced with a stop codon;
the polynucleotide as set forth in SEQ ID NO: 25, wherein a codon of SEQ ID NO: 25 encoding glycine at position 617 of SEQ ID NO: 26 is replaced with glutamic acid;
the polynucleotide as set forth in SEQ ID NO: 27, wherein a codon of SEQ ID NO: 27 encoding tryptophan at position 17 of SEQ ID NO: 28 is replaced with a stop codon;
the polynucleotide as set forth in SEQ ID NO: 27, wherein a codon of SEQ ID NO: 27 encoding glutamine at position 114 of SEQ ID NO: 28 is replaced with a stop codon;
the polynucleotide as set forth in SEQ ID NO: 29, wherein a codon of SEQ ID NO: 29 encoding proline at position 128 of SEQ ID NO: 30 is replaced with leucine;
the polynucleotide as set forth in SEQ ID NO: 29, wherein a codon of SEQ ID NO: 29 encoding proline at position 127 of SEQ ID NO: 30 is replaced with leucine;
the polynucleotide as set forth in SEQ ID NO: 29, wherein a codon of SEQ ID NO: 29 encoding glycine at position 156 of SEQ ID NO: 30 is replaced with serine;
the polynucleotide as set forth in SEQ ID NO: 31, wherein a codon of SEQ ID NO: 31 encoding methionine at position 1 of SEQ ID NO: 32 is replaced with isoleucine;
the polynucleotide as set forth in SEQ ID NO: 31, wherein a codon of SEQ ID NO: 31 encoding glutamine at position 389 of SEQ ID NO: 32 is replaced with a stop codon;
the polynucleotide as set forth in SEQ ID NO: 33, wherein a codon of SEQ ID NO: 33 encoding glutamine at position 465 of SEQ ID NO: 34 is replaced with a stop codon;
the polynucleotide as set forth in SEQ ID NO: 33, wherein a codon of SEQ ID NO: 33 encoding leucine at position 689 of SEQ ID NO: 34 is replaced with phenylalanine;
the polynucleotide as set forth in SEQ ID NO: 35 and further comprising a splice site disruption;
the polynucleotide as set forth in SEQ ID NO: 35, wherein a codon of SEQ ID NO: 35 encoding leucine at position 151 of SEQ ID NO: 36 is replaced with phenylalanine;
the polynucleotide as set forth in SEQ ID NO: 35, wherein a codon of SEQ ID NO: 35 encoding glycine at position 166 of SEQ ID NO: 36 is replaced with aspartic acid;
the polynucleotide as set forth in SEQ ID NO: 35, wherein a codon of SEQ ID NO: 35 encoding threonine at position 200 of SEQ ID NO: 36 is replaced with isoleucine;
the polynucleotide as set forth in SEQ ID NO: 35, wherein a codon of SEQ ID NO: 35 encoding leucine at position 284 of SEQ ID NO: 36 is replaced with phenylalanine;
the polynucleotide as set forth in SEQ ID NO: 35, wherein a codon of SEQ ID NO: 35 encoding leucine at position 300 of SEQ ID NO: 36 is replaced with phenylalanine;
the polynucleotide as set forth in SEQ ID NO: 37, wherein a codon of SEQ ID NO: 37 encoding glutamine at position 961 of SEQ ID NO: 38 is replaced with a stop codon;
the polynucleotide as set forth in SEQ ID NO: 37, wherein a codon of SEQ ID NO: 37 encoding cysteine at position 927 of SEQ ID NO: 38 is replaced with tyrosine;
the polynucleotide as set forth in SEQ ID NO: 37, wherein a codon of SEQ ID NO: 37 encoding arginine at position 873 of SEQ ID NO: 38 is replaced with lysine;
the polynucleotide as set forth in SEQ ID NO: 39, wherein a codon of SEQ ID NO: 39 encoding serine at position 560 of SEQ ID NO: 40 is replaced with leucine;
the polynucleotide as set forth in SEQ ID NO: 39, wherein a codon of SEQ ID NO: 39 encoding glycine at position 424 of SEQ ID NO: 40 is replaced with glutamic acid;
the polynucleotide as set forth in SEQ ID NO: 39, wherein a codon of SEQ ID NO: 39 encoding glutamic acid at position 864 of SEQ ID NO: 40 is replaced with lysine;
the polynucleotide as set forth in SEQ ID NO: 39, wherein a codon of SEQ ID NO: 39 encoding glutamine at position 915 of SEQ ID NO: 40 is replaced with a stop codon; or,
the polynucleotide as set forth in SEQ ID NO: 39, wherein a codon of SEQ ID NO: 39 encoding glycine at position 960 of SEQ ID NO: 40 is replaced with aspartic acid.

17. The plant of claim 13, wherein the plant is Solanum lycopersicum, Capsicum annuum or Cucumis sativa.
